(19) United States
(12) Patent Application Publication
(10) Pub. No.: US 2004/0101522 A1
(43) Pub. Date: May 27, 2004

(54) Transduced neoplastic cell preparations able to express T-cell costimulatory molecules B7.1, ICAM-1, and LFA-3 and induce immunostimulatory prophylactic and therapeutic anti-tumor effects in-vivo

(75) Inventors: Dennis L. Panicali, Acton, MA (US); Ronald Levy, Stanford, CA (US)

Correspondence Address: NIXON PEABODY LLP, 101 FEDERAL ST, BOSTON, MA, 02110, US

(73) Assignees: THERION BIOLOGICS CORPORATION, Cambridge, MA (US); The Board of Trustees of the Leland Stanford Junior University, Palo Alto, CA (US)

(21) Appl. No.: 10/660,369

(22) Filed: Sep. 11, 2003

Related U.S. Application Data
(60) Provisional application No. 60/410,161, filed on Sep. 12, 2002.

(51) Int. Cl. A61K 48/00; C12N 5/08

(52) U.S. Cl. 424/93.21; 435/366

(57) ABSTRACT

The present invention provides a neoplastic cell transduced by a TRICOM viral vector construct and a genetically altered neoplastic cellular preparation able to overexpress molecule B7.1, intracellular adhesion molecule-1 (ICAM-1) and leukocyte function-associated antigen-3 (LFA-3) as discrete products and costimulatory molecules. The TRICOM transduced neoplastic cells are useful in-vivo as a prophylactic vaccination to prevent the future generation of a tumor within a living mammalian subject; and also as a therapeutic anti-tumor agent effective against a pre-existing tumor then located at one or more anatomic sites within the body of a living mammalian patient.

### RESEARCH SUPPORT

The research effort resulting in the present invention was supported in part by grants CA 34233 and 33399 from the U.S. Public Health Service. The U.S. government has certain rights in the invention.

### FIELD OF THE INVENTION

The present invention is concerned generally with compositions of matter and methods for efficaciously activating lymphocytes in-vivo; and is particularly directed to treatment methods against a pre-existing tumor in-vivo as well as prophylactic methods which prevent the generation of a tumor in-vivo in the future.

### BACKGROUND OF THE INVENTION

In recent years, some approaches to increasing the immunogenicity of tumors have involved the transfer of exogenous genes encoding costimulatory molecules. The relevance of costimulation to tumor immunity was demonstrated by a showing that transfection of costimulatory molecules into a subset of murine tumors confers immunogenicity [Dunussi-Joannopoulos et al., Blood 87:2938-2946 (1996); Townsend et al., Science 259:368-370 (1993)].

Molecule B7-1 has been among those stimulatory antigens most extensively studied. Through its interaction with CD28 on T cells, molecule B7-1 is demonstrably able to enhance T-cell activation. However, it is now recognized that at least two other molecules expressed by an antigen presenting cell (an “APC”) are critical for providing T-cell stimulation—i.e., ICAM-1, and LFA-3 [Parra et al., J. Immunol. 158:637-642 (1997); Wingren et al., Crit. Rev. Immunol. 15:235-253 (1995); Camacho et al., Nat. Immunol. 2:523-529 (2001)]. Moreover, these molecules synergize with each other to amplify the activation of T cells [Hodge et al., Cancer Res. 59:5800-5807 (1999); Deeths et al., Eur. J. Immunol. 29:45-53 (1999)].

It is also now known that the state of the antigen presenting cell (APC) significantly affects the degree of T-cell activation. For example, B-cell lymphomas can be induced to function as antigen presenting cells; and as APCs, present tumor antigens directly to T cells to activate them [Bogen, B. and B. Malissen, Eur. J. Immunol. 16:1373-1378 (1986); Bogen, B. and S. Weiss, Int. Rev. Immunol. 10:337-355 (1993)]. In addition, the use of the CD40 ligand increases the immunogenicity of the murine A20 lymphoma, by upregulating costimulatory molecules. This is a concept that has also been recently tested for human B-cell malignancies [Kato et al., J. Clin. Invest. 101:1133-1141 (1998); Viola, A. and A. Lanzavecchia, Science 273:104-106 (1996)]. Yet despite the expression of such relevant antigens, a tumor may nevertheless fail to induce a stimulation of T cells and a cellular immune response if the expression of costimulatory molecules by the tumor is not quantitatively adequate [Liebowitz et al., Curr. Op. Oncol. 10:533-541 (1998)].

Vaccination Therapies

Vaccination with genetically manipulated tumor cells has become an attractive therapeutic approach for patients with tumor malignancies. Since tumor cells have the potential for antigen presentation, approaches that might employ this potential have been considered for use in oncologic immunotherapy. Against this approach and concept, however, is the undisputed fact that tumor cells often fail to active T cells and typically fail to induce T-cell proliferation in vivo within the autologous host [Schultze, J., Leuk. Lymph. 32:223-236 (1999)].

In order to function effectively as an antigen presenting cell (APC), tumor cells must express peptides which not only must become bound to at least one of the major histocompatibility complex (MHC) class I or II molecules, but also must express a number of costimulatory molecules that enhance T-cell activation and proliferation [Liebowitz et al., Curr. Op. Oncol. 10:533-541 (1998)]. Among these, molecule B7-1, intercellular adhesion molecule-1 (ICAM-1), and leukocyte function-associated antigen-3 (LFA-3) have been shown to play pivotal roles in T cell stimulation [Hellstrom et al., Ann. N.Y. Acad. Sci. 690:225-230 (1993)]. Thus the failure of tumor cells to function as an APC has been attributed to and is believed mainly to be due to deficient expression of these three costimulatory molecules [Schultze et al., Proc. Natl. Acad. Sci. 92:8200-9204 (1995)].

However, the mere fact that a cell can express costimulatory molecules on its surface does not necessarily alone determine or even indicate that such a cell will efficaciously activate T cells. Rather, it appears that the capability and power of an APC to activate T cells depends instead on the quantum of expression for these costimulatory molecules at the cell surface [Croft et al., J. Immunol. 152:2675-2685 (1994)]. Thus, different quantum levels in the expression of affect and regulate the immunogenicity of a tumor; and such quantum differences in expression markedly influence the outcome and effectiveness of an antitumor response [Viola, A. and A. Lanzavecchia, Science 273:104-106 (1996)].

For these reasons, despite the range and variety of knowledge acquired to date, the capability to induce a hyperexpression of costimulatory molecules on-demand in a tumor or the capacity to treat a tumor in-vivo within a living subject therapeutically and efficaciously has not been meaningfully achieved or demonstrated to date. Accordingly, were such a development to occur, it would be recognized and appreciated by physicians and oncologists alike as a major medical advance and long-sought treatment regimen.

### SUMMARY OF THE INVENTION

The present invention has multiple aspects and alternative delineations which utilize and employ requisite and essential compositions of matter.

A first aspect and definition is a genetically altered neoplastic cell useful as an immunostimulatory agent against a tumor of interest, said genetically altered neoplastic cell comprising:

a neoplastic cell of mammalian origin which is representative of the cells constituting a tumor of interest;

a genetically altered genome including at least one extra nucleotide segment comprising a viral vector and not less than one DNA sequence encoding molecule B7.1, intracellular adhesion molecule-1 (ICAM-1) and leukocyte function-associated antigen-3 (LFA-3) as specific products;

the capacity to overexpress molecule B7.1, intracellular adhesion molecule-1 (ICAM-1) and leukocyte function-associated antigen-3 (LFA-3) as discrete products; and

the capability to interact with and to activate CD4+ and CD8+ T-cell lymphocytes in-situ.

A second aspect and definition is genetically altered neoplastic cell preparation useful as a prophylactic vaccine in-vivo to prevent the generation of a tumor within the body of a living mammalian subject, said genetically altered neoplastic cell preparation comprising:

a plurality of transduced neoplastic cells of mammalian origin which are representative of the tumor to be prevented within the body of the living mammalian subject and which have the capability to interact with and to activate CD4+ and CD8+ T-cell lymphocytes in-situ, said transduced neoplastic cells

(i) being transduced with a viral vector carrying not less than one DNA sequence encoding molecule B7.1, intracellular adhesion molecule-1 (ICAM-1) and leukocyte function-associated antigen-3 (LFA-3); and

(ii) overexpressing molecule B7.1, intracellular adhesion molecule-1 (ICAM-1) and leukocyte function-associated antigen-3 (LFA-3) as discrete peptides and functional costimulatory molecules.

A third aspect and definition is a genetically altered neoplastic cell preparation useful as a therapeutic anti-tumor agent in-vivo to treat clinically a pre-existing tumor within the body of a living mammalian subject, said genetically altered neoplastic cell preparation comprising:

a plurality of transduced neoplastic cells of mammalian origin which are representative of the cells in the pre-existing tumor within the body of the living mammalian subject and which have the capability to interact with and to activate CD4+ and CD8+ T-cell lymphocytes in-situ, said transduced neoplastic cells

(i) being transduced with a viral vector carrying not less than one DNA sequence encoding molecule B7.1, intracellular adhesion molecule-1 (ICAM-1) and leukocyte function-associated antigen-3 (LFA-3); and

(ii) overexpressing molecule B7.1, intracellular adhesion molecule-1 (ICAM-1) and leukocyte function-associated antigen-3 (LFA-3) as discrete peptides and functional costimulatory molecules.

Other aspects of the subject matter as a whole comprising the present invention include: a method for making a genetically altered neoplastic cell useful as an agent against a tumor of interest; a method for making a transduced neoplastic cell preparation useful as an immunostimulatory agent in-vivo effective against a tumor of interest; a method of in-vivo prophylaxis to prevent the generation of a tumor in a living mammalian subject; and a method of in-vivo therapeutic treatment effective against a pre-existing tumor in a living mammalian subject.

### BRIEF DESCRIPTION OF THE FIGURES

The present invention may be better appreciated and more easily understood when taken in conjunction with the accompanying drawing, in which:

FIGS. 1A-1D are graphs illustrating the upregulation of costimulatory molecules on neoplastic ells after transduction with a TRICOM vector construct;

FIG. 2 is a graph illustrating the survival of mice vaccinated prophylactically with TRICOM-induced tumor cells;

FIGS. 3A and 3B are graphs illustrating the survival of mice with pre-existing tumors which were treated therapeutically with TRICOM-transduced tumor cells;

FIG. 4 is a graph illustrating the consequences of treatment of mice with established tumors with intratumoral injection of recombinant fowlpox viruses;

FIG. 5 is a graph illustrating the cytotoxic T lymphocyte activity induced by TRICOM-transduced tumor cells; and

FIG. 6 is a graph illustrating the effect of in-vivo depletion of T-cell subsets on survival following prophylactic vaccination with TRICOM-transduced tumor cells.

### DETAILED DESCRIPTION OF THE INVENTION

The present invention is a neoplastic cell which has been infected and transduced by a TRICOM viral vector construct; and provides a genetically altered neoplastic cellular preparation able to overexpress molecule B7.1, intracellular adhesion molecule-1 (ICAM-1) and leukocyte function-associated antigen-3 (LFA-3) as discrete products and costimulatory molecules. The TRICOM transduced neoplastic cell and genetically altered cellular preparations are compositions of matter useful in-vivo, in either prophylactic or therapeutic modes of treatment.

The prophylactic method of the invention utilizes the compositions of matter as a vaccine to prevent the future generation of a tumor within the body of a living mammalian subject. In comparison, the therapeutic treatment method of the invention employs these compositions as anti-tumor agents which are effective against a pre-existing tumor which is then located at one or more anatomic sites within the body of a living mammalian patient.

I order to appreciate better what are the unique benefits and advantages offered by the present invention as a whole, the details of the invention are disclosed as different descriptive section presented seriatim hereinafter.

### I. In-Vivo Tumors

The intended objective and target for the present invention is the efficacious treatment (either prophylactically or therapeutically) of in-vivo tumors generally; and for the treatment of neoplastic disorders of the lymphoid tissue in particular—i.e., lymphomas and leukemias as such.

By definition, a lymphoma is a general medical term applied to any neoplastic disorder of the lymphoid tissue, including Hodgkin's disease. An intranodal lymphoma is a tumor arising within the hemopoietic and lymphoid tissues of the body. In comparison, an extranodal lymphoma is a tumor arising in tissues other than the hemopoietic and lymphoid tissues.

Malignant Lymphomas:

By definition, a malignant lymphoma is a solid neoplasm derived from cells of the lymphoreticular system. Malignant lymphomas may be limited to a single anatomic site, particularly lymph nodes; but they may also arise from the spleen, tonsillar tissue of Waldeyer's ring, lymphoid tissues of the gastrointestinal tract; and, in 15-25 percent of all cases, from sites outside the usual lymphoid-containing organs (extranodal lymphomas). Dissemination may occur by involvement of the bone marrow and peripheral blood, at which time the lymphoma may be indistinguishable from leukemia.

Note that lymphomas and leukemias are very much alike as neoplastic (tumor) cells. The basic distinction between lymphomas and leukemia depends on the anatomic distribution of disease and its presentation, and on the evolution of the neoplastic process.

Lymphomas are divided into two major clinicopathologic groups. Hodgkin's disease and the non-Hodgkin's lymphomas. Hodgkin's disease is manifested primarily by lymph node enlargement and may remain localized to one site for a variable length of time. It tends to spread in a predictable manner, involving contiguous lymph node groups and organs. The anatomic distribution of lesions in patients with untreated Hodgkin's disease differs considerably from that found in non-Hodgkin's lymphomas. For example, studies show a relatively low percentage of involvement of the mesenteric nodes and bone marrow as compared with many non-Hodgkin's lymphomas; in contrast, Hodgkin's disease has a higher incidence of mediastinal lymph node involvement.

### Hodgkin's Disease

The diagnosis of Hodgkin's disease is made primarily on the basis of histopathologic changes, and depends on the identification of the Reed-Sternberg cell. Currently thought to be of macrophage/histiocytic lineages, the cell is classically binucleated: the nuclei appear mirror imaged, with large, acidophilic, inclusionlike nucleoli, and are surrounded by a clear perinuclear halo. The recognition of Reed-Sternberg cells is mandatory in the initial diagnosis of Hodgkin's disease, provided that these cells are present in an appropriate stromal environment, which varies depending on the sub-classification.

Currently, the Rye modification of the Lukes and Butler classification is used and is presented by Table 1 below. The classification depends on the number of lymphocytes, which range from many (lymphocytic predominance) to a form in which lymphocytes are rare but Reed-Sternberg cells are remarkably increased (lymphocytic depletion); the mixed cellularity type falls between these two types. The nodular sclerosing type is separate and forms the largest category of Hodgkin's disease. In this form, the lymphoid tissue is divided into nodules by proliferating bands of collagen; Reed-Sternberg cells and variants (lacunar cells) may lie within clear spaces within the lymphoid nodules.

TABLE 1
Rye Classification of Hodgkin's Disease*

Lymphocytic predominance
Nodular sclerosis
Mixed cellularity
Lymphocytic depletion
*Classification modified from Lukes and Butler.

The pathologic subtypes of Hodgkin's disease historically are predictable in their clinical behavior. The nodular sclerosing type is found typically in young females, with a predilection for the mediastanum, including both the lymph nodes and thymus. Such patients generally have a good prognosis.

The lymphocytic predominance form represents about 5 percent of all cases of Hodgkin's disease, is found mainly in children, and has a good prognosis. Conversely, the lymphocytic depletion form has a bad prognosis, and patients often have bulky disease with widespread involvement. The mixed cellularity form falls between the nodular sclerosing type and lymphocyte depletion form in prognosis.

Under modern therapeutic regimens employing both radiation therapy and combination chemotherapy, the subtypes of Hodgkin's disease are losing their importance in terms of prognosis. The clinical stage of the disease (extent and symptomatology) is the paramount indicator for prognosis and type of therapy.

The treatment of Hodgkin's disease has evolved, with radiation therapy used as initial treatment only for patients with low-risk stage IA and IIA disease. Staging is usually clinical, and laparotomy is not routinely performed. The addition of limited chemotherapy for some patients treated with radiation is under study and appears promising.

Most patients with Hodgkin's disease (including all with stage IIIB and IV disease) are best treated today with combination chemotherapy using doxorubicin (Adriamycin), bleomycin, vincristine, and dacarbazine (ABVD). This has been shown to be both more effective and less toxic than mechlorethamine, vincristine, procarbazine, and prednisone (MOPP). In particular, there is less reproductive sterility and less secondary leukemia.

All patients with both localized and disseminated disease should be treated with curative intent. The prognosis of patients with stage IA or IIA disease treated with radiotherapy is excellent, with 10-year survival rates in excess of 80%. Patients with disseminated disease (IIIB, IV) have 5-year survival rates of 50-60%. Poorer results are seen in patients who are older, those who have bulky disease, and those with lymphocyte depletion or mixed cellularity on histologic examination. Patients whose disease recurs after initial radiotherapy treatment may still be curable with chemotherapy. The treatment of choice for patients who relapse after initial chemotherapy is high-dose chemotherapy with autologous stem cell transplantation. This offers a 35-50% chance of cure to patients whose disease is still chemotherapy-sensitive.

### Non-Hodgkin's Lymphomas

The non-Hodgkin's lymphomas are a heterogeneous group of cancers of lymphocytes. The disorders are variable in clinical presentation and course, varying from indolent disease to rapidly progressive devastating illnesses. The non-Hodgkin's lymphomas thus represent a diverse clinical and pathologic group of neoplasms, most of which arise from lymphocytes in various degrees of transformation.

The nomenclature and classification of non-Hodgkin's lymphomas have been the subject of controversy, but the classification given by accompanying Table 2 below has been proposed by the National Cancer Institute to serve as a correlation between the numerous schemes. The formulation divides lymphomas into three prognostic groups, designated as low, intermediate, or high grade, which imply the virulence or degree of malignancy. The fourth group serves as a general catch-all category for all the other types and forms not otherwise suitable for inclusion into the three prognostic categories.

TABLE 2
Classification of Non-Hodgkin's lymphomas

| Grade | Type |
|---|---|
| Low | Small lymphocytic |
| | Small lymphocytic, plasmacytoid |
| | Follicular small cleaved cell |
| | Follicular mixed cell |
| Intermediate | Follicular large cell |
| | Diffuse small cleaved cell |
| | Diffuse mixed cell |
| | Diffuse large cell |
| High | Immunoblastic |
| | Small noncleaved (Burkitt's) |
| | Small noncleaved (non-Burkitt's) |
| | Lymphoblastic |
| | True histiocytic |
| Miscellaneous/Other | Cutaneous T cell (mycosis fungoides) |
| | Adult T cell leukemia- lymphoma |
| | Mantle cell |
| | Marginal zone (MALT) |
| | Peripheral T cell |
| | Anaplastic large cell |

For each prognostic category, there are several histopathologic types based both on the identification of the predominant cell type and the architectural pattern, diffuse or follicular (nodular). For example, individuals with follicular lymphomas have a significantly lower survival than those with the diffuse counterpart, but within the follicular lymphomas the lymphomas composed of larger cells have a worse prognosis. Paradoxically, despite their favorable prognosis, follicular lymphomas show a tendency for early wide dissemination to many sites—including bone marrow, which is reported to be involved in as many as 60 percent of these cases. It has also recently been demonstrated that all lymphomas with a follicular pattern, regardless of cell type, are derived from lymphocytes originating in the follicular or germinal center (B lymphocytes).

The majority of diffuse lymphomas are thus derived predominantly from B lymphocytes. The diffuse large cell lymphomas, for example, originate from both bone marrow-derived and follicular center cell B lymphocytes, as well as thymic-processed (T) lymphocytes. Regardless of origin, this form of lymphoma frequently presents in extranodal sites, and despite apparent localized disease has an aggressive clinical course and prognosis.

Curiously, however, malignant lymphomas in children are different; the working formulation is not strictly applicable. Follicular lymphomas of any type are virtually nonexistent in children and adolescents, as is the small lymphocytic type. The childhood non-Hodgkin's lymphomas are characterized by cells that have primitive cytologic features with a high mitotic index, rapid growth, and a high incidence of mediastinal and extranodal presentations. The lymphoblastic type, for example, commonly presents with mediastinal masses and displays a tendency for early marrow and peripheral blood involvement, at which time the lymphoma is indistinguishable from acute lymphocytic leukemia. In contrast, the small, non-cleaved variety of lymphoma, which is equally common in children, has a tendency for extranodal presentations such as GI tract or jaw, and a lesser tendency for leukemic manifestations.

The conventional treatment of non-Hodgkin's lymphomas depends on the stage of disease and the clinical status of the patient. A small number of patients have limited disease with only one abnormal lymph node. These patients are treated with localized irradiation. Most patients with indolent lymphoma have disseminated disease at the time of diagnosis. If the disease is not bulky and the patient not asymptomatic, no initial therapy may be required.

Some patients will have spontaneous remissions and may defer treatment for 1-3 years. In the past, standard chemotherapy for patients requiring treatment has been based on alkylators such as chlorambucil, 0.6-1 mg/kg every 3 weeks, or combination therapy with cyclophosphamide, vincristine, and prednisone (CV). However, chemotherapy with fludarabine may produce equivalent results. A monoclonal antibody (rituximab) directed against the B cell surface antigen CD20 is very effective as salvage therapy for relapsed low-grade B cell lymphomas and may improve outcomes when added to initial chemotherapy. Occasional young patients with clinically aggressive low-grade lymphomas may be appropriate candidates for allogeneic transplantation. The role of autologous transplantation remains controversial.

Patients with intermediate-grade lymphomas such as diffuse large cell lymphoma are treated with curative intent. Patients with localized disease are treated with short-course chemotherapy (such as three courses of cyclophosphamide, doxorubicin [Adriamycin], vincristine [Oncovin], and prednisone [CHOP] plus localized radiation. Most patients who have more advanced disease are treated with six to eight cycles of chemotherapy such as CHOP. Some patients with high-risk lymphoma are best treated with autologous stem cell transplantation early in their course.

Patients with intermediate-grade lymphoma who relapse after initial chemotherapy may still be cured by autologous stem cell transplantation if their disease is still responsive to chemotherapy.

The median survival of patients with non-Hodgkin's lymphomas is 6-8 years. These diseases ultimately become refractory to chemotherapy. This often occurs at the time of histologic progression of the disease to a more aggressive form of lymphoma.

The International Prognostic Index is now widely used to categorize patients with intermediate grade lymphoma into risk groups. Factors that confer adverse prognosis are age over 60 years, elevated serum LDH, advanced stage disease (stage III or stage IV), and poor performance status. Patients with no risk factors or one risk factor have high complete response rates (80%) to standard chemotherapy, and most responses (80%) are durable. Patients with two risk factors have a 70% complete response rate, and 70% are long-lasting. Patients with higher-risk disease have lower response rates and poor survival with standard regimens, and alternative treatments are needed. Early treatment with high-dose therapy and autologous stem cell transplantation improves the outcome.

For patients who relapse after initial chemotherapy, the prognosis depends on whether the lymphoma is still partially sensitive to chemotherapy. If it is, autologous transplantation offers a 50% chance of long-term salvage.

The treatment of older patients with lymphoma has been difficult because of their poorer tolerance of aggressive chemotherapy. The use of myeloid growth factors to reduce neutropenic complications may improve outcomes.

A range of medical publications and research reports are commonly available which relate to lymphomas generally as well as to Hodgkin's and non-Hodgkin's lymphomas in particular. Representative of such printed publications conventionally available in this art are: Apostolidis J. et al., J. Clin. Oncol. 2000:18-527; Armitage, J O, J. Clin. Oncol. 1998;16-2780; Harris, N L, et al., J. Clin. Oncol. 1999;17:3835; Miller, T P et al., NEJM 1998;339:31; Shipp, M A, et al., J. Clin. Oncol. 1999;17:423; Sweetenham, J W et al., Clin. Oncol. 1999;17:3101; Aisenberg A C, Blood 1999;93:761; Andre M., J. Clin. Oncol. 1999;17:222; Diehl, V, J. Clin. Oncol. 1999;17:776.

### II. Viral Carriers and Viral Vector Constructs

The present invention utilizes a wide range and variety of viruses as recombinant vectors and viral carriers. It is presumed that the practitioner ordinarily skilled in this field is conversant with conventional techniques for growing, maintaining, and genetically modifying viruses as needed, on-demand.

As a point of information also, it will be recognized and appreciated that in terms of preparing and using suitable vector constructs, it is important, if not essential, that the user be at least familiar with the many established procedures and conventionally known techniques for manipulating and modifying DNA (and RNA) fragments as well as the viral vectors to carry them which have been reported and are today widespread in use and application. Merely exemplifying the many authoritative texts and published articles presently available in the literature regarding genes, DNA nucleotide manipulation and the expression of proteins from manipulated DNA fragments are the following: Gene Probes for Bacteria (Macario and De Marcario, editors) Academic Press Inc., 1990; Genetic Analysis, Principles Scope and Objectives by John R. S. Ficham, Blackwell Science Ltd., 1994; Recombinant DNA Methodology II (Ray Wu, editor), Academic Press, 1995; Molecular Cloning, A Laboratory Manual (Maniatis, Fritsch, and Sambrook, editors), Cold Spring Harbor Laboratory, 1982; PCR (Polymerase Chain Reaction), (Newton and Graham, editors), Bios Scientific Publishers, 1994; and the many references individually cited within each of these publications. All of these published texts are expressly incorporated by reference herein.

Viral Vectors

The range of viral vectors and recombinant vector constructions suitable for use in the present invention is very large and variable. These vector constructs and viral carriers presently include: Retrovirus [U.S. Pat. No. 5,662,896]; poliovirus [Evans et al., Nature 339:385-388 (1989) and Sabin, J. Biol. Standardization 1:115-118 (1973)]; rhinovirus; pox viruses, such as canary pox virus or vaccinia virus [Fisher-Hoch et al., PNAS 86:317-321 (1989); Flexner et al., Ann. N.Y. Acad. Sci. 560:86-103 (1989); Flexner et al., Vaccine 8:17-21 (1990); U.S. Pat. Nos. 4,603,112 and 4,769,330]; SV40 [Mulligan et al., Nature 277:108-114 (1979)]; influenza virus [Luytjes et al., Cell 59:1107-1113 (1989); McMichael et al., The New England Journal of Medicine 309:13-17 (1983); and Yap et al., Nature 273:238-239 (1978)]; adenovirus [Beckner, Biotechniques 6:616-627 (1988), and Rosenfeld et al., Science 252:431-434 (1991)]; parvovirus such as adeno-associated virus (Samulski et al., Journal of Virology 63:3822-3828 (1989), and Mendelsohn et al., Virology 166:154-165 (1988); herpes (Kit, Adv. Exp. Med. Biol. 215:219-236 (1989)]; SV40; HIV; measles (EP 9 440,219); and Sindbis virus (xiong et al., Science 234:1188-1191 (1989); and corona virus. In addition, viral carriers may be either homologous, or non-pathogenic (defective), or replication competent virus and nevertheless induce cellular immune responses.

Poxviruses

Although the present invention can employ a wide range of viruses as recombinant vector carriers for infection and transduction of tumor cells, the preferred agents are the entire Order of Poxviruses generally. This Order includes all the presently recognized genera and species conventionally known and characterized as an example of poxviruses in this art.

### A. Characteristics Of Poxviruses Generally

Poxviruses constitute a group of agents that infect both humans and lower animals and produce characteristic vesicular skin lesions, often called pocks. Poxviruses (family Poxyiridae) are the largest of animal viruses; and they can be seen with phase optics or in stained preparations with the light microscope. The viral particles (originally called elementary bodies) are somewhat rounded, brick-shaped, or ovoid, and have a complex structure consisting of an internal central mass, the nucleoid, surrounded by two membrane layers. The surface is covered with ridges which may be tubules or threads. Poxviruses contain DNA, protein, and lipid. They are relatively resistant to inactivation by common disinfectants and by heat, drying, and cold. A listing of the commonly-shared characteristics of the poxviruses is provided by Table 3 below.

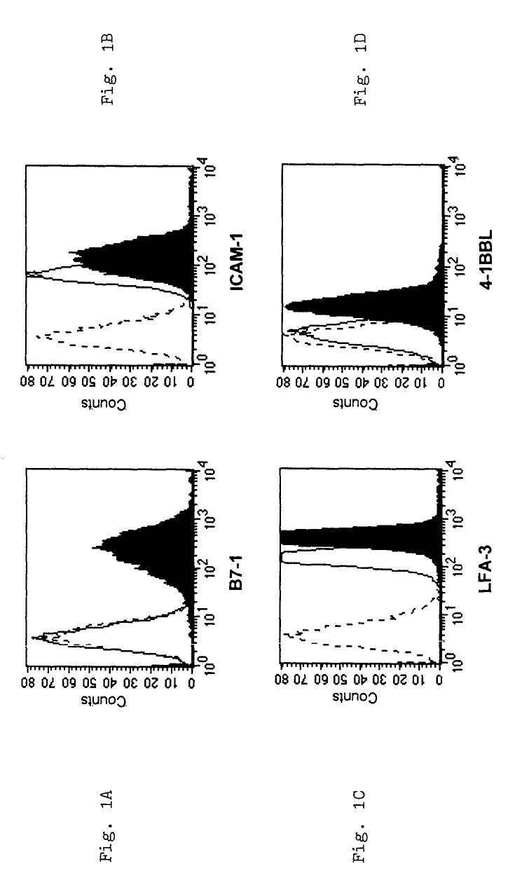

TABLE 3
Characteristics of the Poxviruses

| | |
|---|---|
| Size | 350-390 nm × 200-260 nm |
| Morphology | Brick-shaped to ovoid (FIGS. 56-1, 56-11, |
| | 56-12) |
| Protein and lipid content | Present |
| Stability | Relatively resistant to inactivation by chemicals |
| | (disinfectants) or by heat, cold, or drying; |
| | inactivated by chloroform, variable inactivation |
| | by ether |
| Nucleic acid | Double-stranded DNA* |
| Mol wt and base ratio | Vaccinia DNA = 150 × 10⁶ daltons (231 Kbp); |
| | AT/GC = 1.67 H |
| | Fowlpox DNA = 200 × 10⁶ daltons (307 Kbp); |
| | AT/GC = 1.84 |
| Antigenicity | Common family and genus Ags |
| MultiplicationI | In cytoplasm of cells |
| Cytopathogenicity | Predilection for epidermal cells; eosinophilic |
| | inclusion bodies produced |

Kbp = kilobase pairs.
*Complementary strands of vaccinia DNA are covalently crosslinked at or near the termini. Other poxviruses have not been examined for this structure.
H DNAs of cowpox, rabbitpox, and mousepox are similar.
I A DNA-dependent RNA polymerase in virion.

All poxviruses are related immunologically by a common internal Ag extractable from viral particles by 1 N NaOH. They can be divided into genera on the basis of their more specific Ags, nucleic acid homology, morphology, and natural hosts.

Genera:

The genus Orthopoxvirus consists of viruses of certain mammals, including variola, vaccinia, cowpox, ectromelia of mice, rabbitpox, and monkeypox. Other genera include viruses specific for birds (Avipoxvirus), ungulates (Capripoxvirus), and arthropods (Entomopoxvirus), and the tumor-producing (fibroma and myxoma) viruses of rabbits (Leporipoxvirus).

Viruses of a sixth genus which resemble other poxviruses in structure but not immunologically (hence termed parapoxviruses) include contagious pustular dermatitis (orf), paravaccinia (milker's modules), and bovine papular stomatitis viruses. Some poxviruses, such as the molluscum contagiosum virus and Yaba monkey tumor virus cannot be classified immunologically in any of these genera.

Poxviruses vary widely in their native ability to cause generalized infection, but they share a predilection for epidermal cells, in which they multiply in the cytoplasm and produce eosinophilic inclusion bodies (termed Guarnieri bodies). Fibroma and myxoma viruses have, in addition, a great affinity for subcutaneous connective tissues. Most poxviruses also multiply readily in epidermal cells of the chorioallantois of chick embryos, where they produce characteristic nodular focal lesions, termed pocks. These lesions reflect a second characteristic of poxviruses: the propensity to cause cellular hyperplasia before cell necrosis. With myxoma, fibroma, and Yaba viruses the hyperplasia predominates, and tumors develop.

### B. Recombinant Poxviruses Suitable as Viral Vectors

Many species of poxviruses have been adapted as recombinant DNA viruses; and have been employed as recombinant viral vectors for the introduction and expression of exogenous DNA in living cells. Of particular interest and value to the present invention are the modified recombinant vaccinia viruses and the means for introducing exogenous DNA into the vaccinia genome as described by U.S. Pat. No. 4,603,112; as well as the recombinant fowlpox virus and means for introducing foreign DNA into the fowlpox genome as described by U.S. Pat. No. 5,093,258. Each of these issued patents is expressly incorporated by reference herein.

In addition, the genera and species of the Order Poxvirus have also been adapted and modified for use as recombinant DNA viral vectors to introduce a variety of exogenous DNA into an infected host cell. Merely examplifying and illustrated the conventionally available range and variety of poxviruses recombinant vectors are the following: a recombinant avipox virus [U.S. Pat. Nos. 5,286,639 and 6,340,462]; a malaria recombinant poxvirus vaccine [U.S. Pat. Nos. 5,756,101 and 6,214,353]; methods using a modified vaccinia virus [U.S. Pat. No. 5,972,597]; recombinant poxvirus compositions and methods of inducing immune responses [U.S. Pat. No. 5,942,235]; a recombinant vaccinia virus expressing human retrovirus gene [U.S. Pat. No. 5,778,210]; Marek's disease virus recombinant poxvirus vaccine [U.S. Pat. Nos. 5,759,553 and 5,759,552]; recombinant vaccina virus encoding HSV glycoproteins [U.S. Pat. No. 5,583,028]; recombinant avipox virus and method to induce an immune response [U.S. Pat. Nos. 5,174,993 and 5,505,941]; a poxvirus insertion/expression vector [U.S. Pat. No. 5,443,964]; a vector for recombinant poxvirus expressing rabies virus glycoproteins [U.S. Pat. No. 5,348,741]; vaccina virus encoding herpes virus glycoproteins [U.S. Pat. No. 5,338,683]; a recombinant fowlpox virus [U.S. Pat. No. 5,180,675]; and a recombinant MVA vaccine virus [U.S. Pat. No. 5,185,146]. The texts of each of these issued patents is also expressly incorporated by reference herein.

### C. Representative Modes of Poxvirus Vector Preparation

The range and variety of poxviruses which can be modified and prepared for use as recombinant viral vectors is very large indeed. For this reason, a number of specific vector preparation techniques and resulting recombinant vectors are described hereinafter. It will be expressly understood, however, that the examples provided are merely representative of the recombinant poxvirus vectors possible; and that these examples are the preferred embodiments illustrative of the far greater range of formats which are potentially possible and useful as vectors within the present invention.

### III. The TRICOM Viral Vector Construct

The preferred recombinant poxvirus vector is constructed to include at least three structural foreign DNA sequences as part of the recombinant viral genome. The requisite exogenous structural DNA coding segments are: a gene or DNA sequence encoding a form of molecule B7.1; DNA or a gene encoding a form of intracellular adhesion molecule-1 (ICAM-1); and DNA or a gene encoding a form of leukocyte function-associated antigen (LFA-3). These three DNA genes are collectively termed “TRICOM” herein. Moreover, each of these three different and individual DNA segments (or genes) must be operatively positioned within the construct to be under the transcriptional control of a promote (or promoter gene) also situated within the viral vector.

It is preferable that each exogenous structural DNA segment (or gene) be compatible with the origin of the tumor cell to be subsequently infected and transduced. Thus, if the source of the tumor cell is from a mouse, then the form of the structural DNA encoding B7.1, ICAM-1 and LFA-3 should be of murine origin. Similarly, if the tumor cell to be transduced is from a human subject, then the DNA (genes) encoding the B7.1, ICAM-1 and LFA-3 segments should also be of human type and human origin DNA sequences.

Lastly, it is permissible—and often quite desirable—that other exogenous DNA segments or foreign structural genes also be included within the poxvirus vector construct. Thus foreign DNA sequences encoding the thymidine kinase (TK) gene; or encoding the MUC-1 gene; or encoding the TAA gene—are individually and collectively acceptable for inclusion with the TRICOM DNA (or genes) in each vector construction.

To demonstrate the mode and manner in which the TRICOM viral vector may be constructed, the following examples are provided as illustrative and representative embodiments.

### EXAMPLE 1

### Generation of Recombinant Vaccinia, rV-TRICOM(mu1) No. vT171

The origin of vaccinia parental virus is the New York City Board of Health strain and was obtained by Wyeth from the New York City Board of Health and passaged in calves to create the Smallpox Vaccine Seed. Flow Laboratories received a lyophilized vial of the Smallpox Vaccine Seed, Lot 3197, Passage 28 from Drs. Chanock and Moss (National Institutes of Health). This seed virus was ether-treated and plaque-purified three times.

For the generation of rV-TRICOM(mu1), a plasmid vector, designated pT5032 was constructed to direct insertion of the foreign sequences into the M2L (30K) gene, which is located in the Hind III M region of the vaccinia genome. The murine LFA-3 gene is under the transcriptional control of the vaccinia 30K (M2L) promoter (ref. 15), the murine ICAM-1 gene is under the control of the vaccinia I3 promoter (ref. 14) and the murine B7.1 gene is under the control of the synthetic early/late (sE/L) promoter (ref. 13). These foreign sequences are flanked by DNA sequences from the Hind III M region of the vaccinia genome. These flanking sequences include the vaccinia K1L host range gene. A derivative of the Wyeth strain of vaccinia was used as the parental virus in the construction of recombinant vaccinia virus. This parental virus, designated vTBC33, lacks a functional K1L gene and thus cannot efficiently replicate on rabbit kidney RK₁₃ cells (ref. 16). The generation of recombinant vaccinia virus was accomplished via homologous recombination between vaccinia sequences in the vTBC33 vaccinia genome and the corresponding sequences in pT5032 in vaccinia-infected RK₁₃ cells transfected with pT5032. Recombinant virus, designated vTl71, was selected by growth on RK₁₃ cells (ATCC, CCL 37). Plaques were picked from the cell monolayer and their progeny were further propagated. Two rounds of plaque isolation and replating on RK₁₃ cells resulted in the purification of the desired recombinant.

### EXAMPLE 2

### Generation of Recombinant Vaccinia, rV-TRICOM(mu2) No. vT199

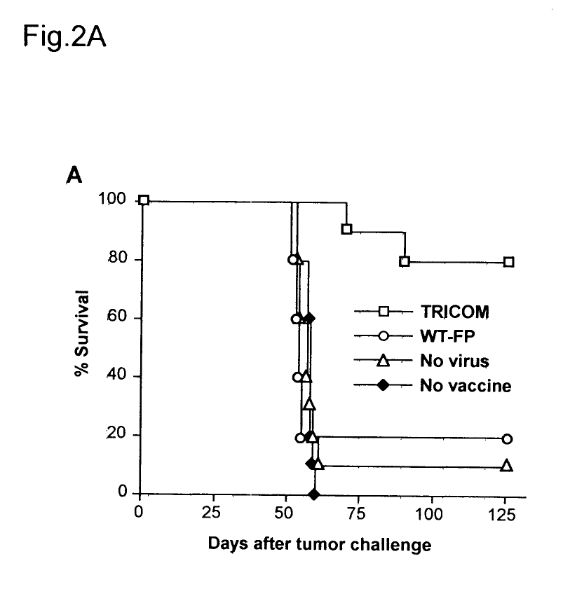

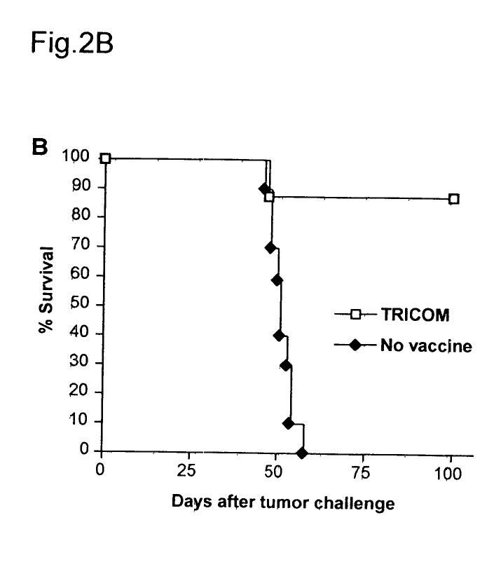

For the generation of rV-TRICOM(mu2), a plasmid vector, designated pT5047, was constructed to direct insertion of the foreign sequences into the thymidine kinase (TK) gene, which is located in the Hind III J region of the vaccinia genome. The murine B7.1 gene is under the control of the sElL promoter, the murine LFA-3 gene is under the transcriptional control of the 13 promoter, and the murine ICAM-1 gene is under the control of the vaccinia 7.5K promoter (ref. 17). In addition, the E. coli lacZ gene, under the control of the fowlpox virus C1 promoter (ref. 7), is included as a screen for recombinant progeny. These foreign sequences are flanked by DNA sequences from the Hind III J region of the vaccinia genome (see FIG. 2). A plaque-purified isolate from the Wyeth (New York City Board of Health) strain of vaccinia was used as the parental virus for this recombinant vaccine. The generation of recombinant vaccinia virus was accomplished via homologous recombination between vaccinia sequences in the Wyeth vaccinia genome and the corresponding sequences in pT5047 in vaccinia-infected Hu143TK-cells (Bacchetti and Graham 1977) transfected with pT5047. Recombinant virus was identified using selection for TK virus in the presence of bromodeoxyuridine (BudR) in combination with a chromogenic assay, performed on viral plaques in situ, that detects expression of the lacZ gene product in the presence of halogenated indolyl-beta-D-galactoside (Bluo-gal), as described previously (ref. 12). Viral plaques expressing lacZ appeared blue against a clear background. Positive plaques, designated vT199, were picked from the cell monolayer and their progeny were replated under the selective conditions described above. In other recombinant viruses selected and purified in this manner, the only plaques that appeared under these selective conditions were blue, and these blue plaques were readily isolated and purified. However, in the case of vT199, only white plaques were observed at the second round of plaque-purification; no blue plaques were apparent. A new set of blue plaques were picked and replated; again, only white plaques were observed at the second round of plaque-purification. A final attempt, using yet another set of blue plaques, yielded both blue and white plaques after the second round of plaque-purification. Blue plaques were selected and replated. Two additional rounds of plaque-purification (a total of four rounds) yielded recombinant viruses that were 100% blue.

### EXAMPLE 3

### Generation of Recombinant Vaccinia rV-TAA/TRICOM(mu)

For the generation of rV-T AA/TRICOM(mu), a plasmid vector is constructed to direct insertion of the foreign sequences into the vaccinia genome. The TAA gene, the murine LFA-3 gene, the murine ICAM-1 gene, and the murine B7.1 gene are under the control of a multiplicity of promoters. These foreign sequences are flanked by DNA sequences from the vaccinia genome, into which the foreign sequences are to be inserted. The generation of recombinant vaccinia virus is accomplished via homologous recombination between vaccinia sequences in the vaccinia genome and the corresponding sequences in the plasmid vector in vaccinia-infected cells transfected with the plasmid vector. Recombinant plaques are picked from the cell monolayer under selective conditions and their progeny are further propagated. Additional rounds of plaque isolation and replating result in the purification of the desired recombinant virus.

### EXAMPLE 4

### Generation of Recombinant Vaccinia rV-MUC-1/TRICOM(mu)

For the generation of rV-MUC-1/TRICOM(mu), a plasmid vector is constructed to direct insertion of the foreign sequences into the vaccinia genome. The MUC-1 gene, the murine LFA3 gene, the murine ICAM-1 gene, and the murine B7.1 gene are under the control of a multiplicity of promoters. These foreign sequences are flanked by DNA sequences from the vaccinia genome into which the foreign sequences are to be inserted. The generation of recombinant vaccinia virus is accomplished via homologous recombination between vaccinia sequences in the vaccinia genome and the corresponding sequences in the plasmid vector in vaccinia-infected cells transfected with the plasmid vector. Recombinant plaques are picked from the cell monolayer under selective conditions and their progeny are further propagated. Additional rounds of plaque isolation and replating result in the purification of the desired recombinant virus.

### EXAMPLE 5

### Generation of Recombinant Vaccinia rV-CEA/TRICOM(mu) No. vT172

For the generation of rV-CEA/TRICOM(mu), a plasmid vector, designated pT5031, was constructed to direct insertion of the foreign sequences into the M2L (30K) gene, which is located in the Hind III M region of the vaccinia genome. The CEA gene is under the control of the 40K promoter (ref. 5), the murine LFA-3 gene is under the control of the 30K promoter, the murine ICAM-1 gene is under the control of the I3 promoter, and the murine B7.1 gene is under the control of the sE/L promoter. These foreign sequences are flanked by DNA sequences from the Hind III M region of the vaccinia genome, including the vaccinia KIL host range gene. vTBC33, described above, was used as the parental virus in the construction of the recombinant vaccinia virus. The generation of recombinant vaccinia virus was accomplished via homologous recombination between vaccinia sequences in the vTBC33 vaccinia genome and the corresponding sequences in pT5031 in vaccinia-infected RK₁₃ cells transfected with pT5031. Recombinant virus, designated vT172, was selected by growth on RK₁₃ cells as described above. Plaques were picked from the cell monolayer and their progeny were further propagated. Two rounds of plaque isolation and replating on RK₁₃ cells resulted in the purification of the desired recombinant.

### EXAMPLE 6

### Generation of Recombinant Fowlpox, rF-TRICOM(mu) No. vT222

The origin of parental fowlpox virus used for the generation of recombinants was plaque-purified from a vial of an USDA-licensed poultry vaccine, POXVAC-TC, which is manufactured by Schering-Plough Corporation. The starting material for the production of POXVAC-TC was a vial of Vineland Laboratories' chicken embryo origin Fowlpox vaccine, obtained by Schering-Plough. The virus was passaged twice on the chorioallantoic membrane of chicken eggs to produce a master seed virus. The master seed virus was passaged 27 additional times in chicken embryo fibroblasts to prepare the POXVAC-TC master seed. To prepare virus stocks for the generation of POXVAC-TC product lots, the POXVAC-TC master seed was passaged twice on chicken embryo fibroblasts. One vial of POXVAC-TC, Serial # 96125, was plaque-purified three times on primary chick embryo dermal cells.

For the generation of rF-TRICOM(mu), a plasmid vector, designated pT8001, was constructed to direct insertion of the foreign sequences into the BamHI J region of the fowlpox genome. The murine B7.1 gene is under the control of the sE/L promoter, the murine LFA-3 gene is under the control of the I3 promoter, the murine ICAM-1 gene is under the control of the 7.5K promoter, and the lacZ gene is under the control of the C1 promoter. These foreign sequences are flanked by DNA sequences from the BamHI J region of the fowlpox genome. A plaque-purified isolate from the POXVAC-TC (Schering-Plough Corporation) strain of fowlpox was used as the parental virus for this recombinant vaccine. The generation of recombinant fowlpox virus was accomplished via homologous recombination between fowlpox sequences in the fowlpox genome and the corresponding sequences in pT8001 in fowlpox-infected primary chick embryo dermal cells transfected with pT8001. Recombinant virus was identified using the chromogenic assay for the lacZ gene product described above. Viral plaques expressing lacZ appeared blue against a clear background. Positive plaques, designated vT222, were picked from the cell monolayer and their progeny were replated. Six rounds of plaque isolation and replating in the presence of Bluo-Gal resulted in the purification of the desired recombinant.

### EXAMPLE 7

### Generation of Recombinant Fowlpox rF-TAA/TRICOM(mu)

For the generation of rF-TAA/TRICOM(mu), a plasmid vector is constructed to direct insertion of the foreign sequences into the BamHI J region of the fowlpox genome. The TAA gene, the murine LFA-3 gene, the murine ICAM-1 gene, and the murine B7.1 gene are under the control of a multiplicity of promoters. In addition, the lacZ gene is under the control of the C1 promoter. These foreign sequences are flanked by DNA sequences from the BamHI J region of the fowlpox genome. A plaque-purified isolate from the POXVAC-TC (Schering-Plough Corporation) strain of fowlpox is used as the parental virus for this recombinant vaccine. The generation of recombinant fowlpox virus is accomplished via homologous recombination between fowlpox sequences in the fowlpox genome and the corresponding sequences in the plasmid vector in fowlpox-infected primary chick embryo dermal cells transfected with the plasmid vector. Recombinant virus is identified using the chromogenic assay for the lacZ gene product described above. Viral plaques expressing lacZ appear blue against a clear background. Positive plaques are picked from the cell monolayer and their progeny are replated. Additional rounds of plaque isolation and replating in the presence of Bluo-Gal result in the purification of the desired virus.

### EXAMPLE 8

### Generation of Recombinant Fowlpox rF-MUC-1/TRICOM(mu)

For the generation of rF-MUC-1/TRICOM(mu), a plasmid vector is constructed to direct insertion of the foreign sequences into the BamHI J region of the fowlpox genome. The MUC-1 gene, the murine LFA-3 gene, the murine ICAM-1 gene, and the murine B7.1 gene are under the control of a multiplicity of promoters. In addition, the lacZ gene is under the control of C1 promoter. These foreign sequences are flanked by DNA sequences from the BamHI J region of the fowlpox genome. A plaque-purified isolate from the POXVAC-TC (Schering-Plough Corporation) strain of fowlpox is used as the parental virus for this recombinant vaccine. The generation of recombinant fowlpox virus is accomplished via homologous recombination between fowlpox sequences in the fowlpox genome and the corresponding sequences in the plasmid vector in fowlpox-infected primary chick embryo dermal cells transfected with the plasmid vector. Recombinant virus is identified using the chromogenic assay for the lacZ gene product described above. Viral plaques expressing lacZ appear blue against a clear background. Positive plaques are picked from the cell monolayer and their progeny are replated. Additional rounds of plaque isolation and replating in the presence of Bluo-Gal result in the purification of the desired recombinant virus

### EXAMPLE 9

### Generation of Recombinant Fowlpox, rF-CEA/TRICOM(mu) No. vT194

For the generation of rF-CEA/TRICOM(mu), a plasmid vector, designated pT5049, was constructed to direct insertion of the foreign sequences into the BamHI J region of the fowlpox genome. The CEA gene is under the control of the vaccinia 40K promoter, the murine B7-1 gene is under the control of the sE/L promoter, the murine LFA-3 gene is under the transcriptional control of the I3 promoter, the murine ICAM-1 gene is under the transcriptional control of the vaccinia 7.5K promoter, and the lacZ gene is under the control of the Cl promoter. These foreign sequences are flanked by DNA sequences from the BamHI J region of the fowlpox genome. A plaque-purified isolate from the POXVAC-TC (Schering Corporation) strain of fowlpox was used as the parental virus for this recombinant vaccine. The generation of recombinant fowlpox virus was accomplished via homologous recombination between fowlpox sequences in the fowlpox genome and the corresponding sequences in pT5049 in fowlpox-infected primary chick embryo dermal cells transfected with pT5049. Recombinant virus was identified using the chromogenic assay for the lacZ gene product described above. Viral plaques expressing lacZ appeared blue against a clear background. Positive plaques, designated vT194, were picked from the cell monolayer and their progeny were replated. Five rounds of plaque isolation and replating in the presence of Bluo-Gal resulted in the purification of the desired recombinant.

### EXAMPLE 10

### Generation of Recombinant Vaccinia, rV-TRICOM (hu) No. vT224

For the generation of rV-TRICOM(hu), a plasmid vector, designated pT5064, was constructed to direct insertion of the foreign sequences into the thymidine kinase (TK) gene, which is located in the Hind III J region of the vaccinia genome. The human LFA-3 gene is under the control of the 30K promoter, the human ICAM-1 gene is under the control of the I 3 promoter, and the human B7.1 gene is under the control of the sE/L promoter. In addition, the E. coli lacZ gene, under the control of the C1 promoter, is included as a screen for recombinant progeny. These foreign sequences are flanked by DNA sequences from the Hind III J region of the vaccinia genome. A plaque-purified isolate from the Wyeth (New York City Board of Health) strain of vaccinia was used as the parental virus for this recombinant vaccine. The generation of recombinant vaccinia virus was accomplished via homologous recombination between vaccinia sequences in the Wyeth vaccinia genome and the corresponding sequences in pT5064 in vaccinia-infected CV-1 cells (ATTC, CCL 70) transfected with pT5064. Recombinant virus was identified using the chromogenic assay for the lacZ gene product described above. Viral plaques expressing lacZ appeared blue against a clear background. Positive plaques, designated vT224, were picked from the cell monolayer and their progeny were replated. Five rounds of plaque isolation and replating in the presence of Bluo-Gal resulted in the purification of the desired recombinant.

### EXAMPLE 11

### Generation of Recombinant Vaccinia rV-TAA/TRICOM(hu)

For the generation of rV-TAA/TRICOM(hu), a plasmid vector is constructed to direct insertion of the foreign sequences into the thymidine kinase (TK) gene, which is located in the Hind III J region of the vaccinia genome. The TAA gene, the human LFA-3 gene, the human ICAM-1 gene, the human B7.1 gene, and the E. coli lacZ gene are under the control of a multiplicity of poxvirus promoters. These foreign sequences are flanked by DNA sequences from the Hind III J region of the vaccinia genome. A plaque-purified isolate from the Wyeth (New York City Board of Health) strain of vaccinia is used as the parental virus for this recombinant vaccine. The generation of recombinant vaccinia virus is accomplished via homologous recombination between vaccinia sequences in the Wyeth vaccinia genome and the corresponding sequences in the plasmid vector in vaccinia-infected CV-1 cells (ATTC, CCL 70) transfected with the plasmid. Recombinant virus is identified using the chromogenic assay for the lacZ gene product described above. Viral plaques expressing lacZ appear blue against a clear background. Positive plaques are picked from the cell monolayer and their progeny are replated. Additional rounds of plaque isolation and replating in the presence of Bluo-Gal result in the purification of the desired recombinant.

### EXAMPLE 12

### Generation of Recombinant Fowlpox rF-TAA/TRICOM(hu)

For the generation of rF-TAA/TRICOM(hu), a plasmid vector is constructed to direct insertion of the foreign sequences into the BamHI J region of the fowlpox genome. The TAA gene, the human LFA-3 gene, the human ICAM-1 gene, the human B7.1 gene, and the E. coli lacZ gene are under the control of a multiplicity of poxvirus promoters. These foreign sequences are flanked by DNA sequences from the BamHI J region of the fowlpox genome. A plaque-purified isolate from the POXVAC-TC (Schering-Plough Corporation) strain of fowlpox is used as the parental virus for this recombinant vaccine. The generation of recombinant fowlpox virus is accomplished via homologous recombination between fowlpox sequences in the fowlpox genome and the corresponding sequences in the plasmid vector in fowlpox-infected primary chick embryo dermal cells transfected with the plasmid vector. Recombinant virus is identified using the chromogenic assay for the lacZ gene product described above. Viral plaques expressing lacZ appear blue against a clear background. Positive plaques are picked from the cell monolayer and their progeny are replated. Additional rounds of plaque isolation and replating in the presence of Bluo-Gal result in the purification of the desired recombinant virus.

### EXAMPLE 13

### Generation of Recombinant Vaccinia Virus, rV-CEA (6D)/TRICOM(hu) No. vT238

For the generation of rV-CEA(6D)/TRICOM(hu), a plasmid vector, designated pT8016, was constructed to direct insertion of the foreign sequences into the thymidine kinase (TK) gene, which is located in the Hind III J region of the vaccinia genome. The CEA gene was altered by in vitro mutagenesis to express full-length protein containing one modified epitope. This mutation changed the encoded amino acid at position 576 from asparagine to aspartic acid. The modified gene, designated CEA(6D), was designed to enhance the immunogenicity of CEA [Zaremba et al, 1997, Cancer Res. 57:4570-4577]. The CEA(6D) gene is under the control of the 40K promoter. The human LFA-3 gene is under the control of the 30K promoter, the human ICAM-1 gene is under the control of the I3 promoter, and the human B7.1 gene is under the control of the sE/L promoter. In addition, the E. coli lacZ gene, under the control of the C1 promoter, is included as a screen for recombinant progeny. These foreign sequences are flanked by DNA sequences from the Hind III J region of the vaccinia genome. A plaque-purified isolate from the Wyeth (New York City Board of Health) strain of vaccinia was used as the parental virus for this recombinant vaccine. The generation of recombinant vaccinia virus was accomplished via homologous recombination between vaccinia sequences in the Wyeth vaccinia genome and the corresponding sequences in pT8016 in vaccinia-infected CV-1 cells (American Type Culture Collection (ATCC, Rockville, Md., CCL 70) transfected with pT8016. Recombinant virus was identified using the chromogenic assay for the lacZ gene product described above. Viral plaques expressing lacZ appeared blue against a clear background. Positive plaques, designated vT238, were picked from the cell monolayer and their progeny were replated. Six rounds of plaque isolation and replating in the presence of Bluo-Gal resulted in the purification of the desired recombinant.

### EXAMPLE 14

### Generation of Recombinant Fowlpox Virus, rF-TRICOM(mu) No. vT251

For the generation of rF-TRICOM(mu), a plasmid vector, designated pT8019, was constructed to direct insertion of the foreign sequences into the BamHI J region of the fowlpox genome. The murine LFA-3 gene is under the control of the 30K promoter, the murine ICAM-1 gene is under the control of the I3 promoter, the murine B7.1 gene is under the control of the sE/L promoter, and the lacZ gene is under the control of the C1 promoter. These foreign sequences are flanked by DNA sequences from the BamHI J region of the fowlpox genome. A plaque-purified isolate from the POXVAC-TC (Schering-Plough Corporation) strain of fowlpox was used as the parental virus for this recombinant vaccine. The generation of recombinant fowlpox virus was accomplished via homologous recombination between fowlpox sequences in the fowlpox genome and the corresponding sequences in pT8019 in fowlpox-infected primary chick embryo dermal cells transfected with pT8019. Recombinant virus was identified using the chromogenic assay for the lacZ gene product described above. Viral plaques expressing lacZ appeared blue against a clear background. Positive plaques, designated vT251, were picked from the cell monolayer and their progeny were replated. Three rounds of plaque isolation and replating in the presence of Bluo-Gal resulted in the purification of the desired recombinant.

### EXAMPLE 15

### Generation of Recombinant Fowlpox Virus, rF-TRICOM(hu) No. vT232

For the generation of rF-TRICOM(hu), a plasmid vector, designated pT5072, was constructed to direct insertion of the foreign sequences into the BamHI J region of the fowlpox genome. The human LFA-3 gene is under the control of the 30K promoter, the human ICAM-1 gene is under the control of the I3 promoter, the human B7.1 gene is under the control of the sE/L promoter, and the lacZ gene is under the control of the C1 promoter. These foreign sequences are flanked by DNA sequences from the BamHI J region of the fowlpox genome. A plaque-purified isolate from the POXVAC-TC (Schering-Plough Corporation) strain of fowlpox was used as the parental virus for this recombinant vaccine. The generation of recombinant fowlpox virus was accomplished via homologous recombination between fowlpox sequences in the fowlpox genome and the corresponding sequences in pT5072 in fowlpox-infected primary chick embryo dermal cells transfected with pT5072. Recombinant virus was identified using the chromogenic assay for the lacZ gene product described above. Viral plaques expressing lacZ appeared blue against a clear background. Positive plaques, designated vT232 were picked from the cell monolayer and their progeny were replated. Four rounds of plaque isolation and replating in the presence of Bluo-Gal resulted in the purification of the desired recombinant.

### EXAMPLE 16

### Generation of Recombinant Fowlpox Virus, rF-MUC-1/TRICOM(mu) No. vT250

For the generation of rF-MUC-1/TRICOM(mu), a plasmid vector, designated pT8020, was constructed to direct insertion of the foreign sequences into the BamHI J region of the fowlpox genome. A truncated version of the MUC-1 gene was used, consisting of the signal sequence, ten copies of the tandem repeat sequence, and the 3′ unique coding sequence. The nucleotide sequence of the tandem repeat region was altered to minimize homology between the repeats without changing the amino acid sequence. The gene was contained on an 1881 bp fragment which includes the truncated coding sequence, 6 nucleotides of the 5′ untranslated region, and 186 nucleotides of the 3′ untranslated region [Gendler et al, 1990, J. Biol. Chem. 265:15286-15293]. The murine LFA-3 gene is under the control of the 30K promoter, the murine ICAM-1 gene is under the control of the I3 promoter, the murine B7.1 gene is under the control of the sE/L promoter, and the lacZ gene is under the control of the C1 promoter. These foreign sequences are flanked by DNA sequences from the BamHI J region of the fowlpox genome. A plaque-purified isolate from the POXVAC-TC (Schering-Plough Corporation) strain of fowlpox was used as the parental virus for this recombinant vaccine. The generation of recombinant fowlpox virus was accomplished via homologous recombination between fowlpox sequences in the fowlpox genome and the corresponding sequences in pT8020 in fowlpox-infected primary chick embryo dermal cells transfected with pT8020. Recombinant virus was identified using the chromogenic assay for the lacZ gene product described above. Viral plaques expressing lacZ appeared blue against a clear background. Positive plaques, designated vT250, were picked from the cell monolayer and their progeny were replated. Four rounds of plaque isolation and replating in the presence of Bluo-Gal resulted in the purification of the desired recombinant.

### EXAMPLE 17

### Generation of Recombinant Fowlpox Virus, rF-CEA(6D)/TRICOM(hu) No. vT236

For the generation of rF-CEA(6D)/TRICOM(hu), a plasmid vector, designated pT2187, was constructed to direct insertion of the foreign sequences into the BamHI J region of the fowlpox genome. The CEA(6D) gene is under the control of the 40K promoter. The human LFA-3 gene is under the control of the 30K promoter, the human ICAM-1 gene is under the control of the I3 promoter, the human B7.1 gene is under the control of the sE/L promoter, and the lacZ gene is under the control of the Cl promote. These foreign sequences are flanked by DNA sequences from the BamHI J region of the fowlpox genome. A plaque-purified isolate from the POXVAC-TC (Schering-Plough Corporation) strain of fowlpox was used as the parental virus for this recombinant vaccine. The generation of recombinant fowlpox virus was accomplished via homologous recombination between fowlpox sequences in the fowlpox genome and the corresponding sequences in pT2187 in fowlpox-infected primary chick embryo dermal cells transfected with pT2187. Recombinant virus was identified using the chromogenic assay for the lacZ gene product described above. Viral plaques expressing lacZ appeared blue against a clear background. Positive plaques, designated vT236, were picked from the cell monolayer and their progeny were replated. Eight rounds of plaque isolation and replating in the presence of Bluo-Gal resulted in the purification of the desired recombinant.

### EXAMPLE 18

### Generation of Recombinant MVA Virus, rMVA-TRICOM(mu) No. vT264

Modified Vaccinia Ankara (MVA) is an attenuated derivative of the Ankara strain of vaccinia virus [Meyer et al, 1991, J. Gen. Virol. 72:1031-1038]. The seed stock from the MVA vaccine used as smallpox vaccine in humans was obtained from Dr. Anton Mayr (Institute for Medical Microbiology, Munich). The seed stock was plaque-purified two times on primary chick embryo dermal cells.

For the generation of rMVA-TRICOM(mu), a plasmid vector, designated pT5085, was constructed to direct insertion of the foreign sequences into the deletion m region of the MVA genome [Meyer et al, 1991, J. Gen. Virol. 72:1031-1038]. The murine LFA-3 gene is under the control of the 30K promoter, the murine ICAM-1 gene is under the control of the I3 promoter, the murine B7.1 gene is under the control of the sE/L promoter, and the lacZ gene is under the control of the C1 promoter. These foreign sequences are flanked by DNA sequences from the deletion III region of the MVA genome. A plaque-purified isolate from the MVA vaccine seed stock was used as the parental virus for this recombinant vaccine. The generation of recombinant MVA was accomplished via homologous recombinant between MVA sequences in the MVA genome and the corresponding sequences in pT5085 in MVA-infected primary chick embryo dermal cells transfected with pT5085. Recombinant virus was identified using the chromogenic assay for the lacZ gene product described above. Viral plaques expressing lacZ appeared blue against a clear background. Positive plaques, designated vT264 were picked from the cell monolayer and their progeny were replated. Four rounds of plaque isolation and replating in the presence of Bluo-Gal resulted in the purification of the desired recombinant.

### EXAMPLE 19

### Recombinant Poxviruses

The individual recombinant vaccinia viruses containing either the gene encoding murine costimulatory molecule B7-1 (designated rV-B7-1) or the gene encoding murine Intercellular adhesion molecule-1 (designated rV-ICAM-1) have been described (refs. 2 and 3). The recombinant vaccinia virus containing the gene for murine CD48 [designated rV-LFA-3; murine CD48 is the homologue of human LFA-3 (CD58) (ref. 1)] was constructed in a similar fashion to rV-B7-1 and rV-ICAM-1, and has been described (ref. 4). In each of these single recombinant vaccinia viruses, the gene encoding the costimulatory molecule was put under the control of the vaccinia virus early/late 40K promoter (ref. 7), and the transgene was inserted into the Hind III M region of the genome of the Wyeth strain of vaccinia virus as described (ref. 5). Recombinant fowlpox viruses were constructed by the insertion of foreign sequences into the BamHI J region of the genome of the POXVAC-TC (Schering Corporation) strain of fowlpox virus as described (ref. 6). In recombinant viruses containing a single foreign gene, the gene is under control of the vaccinia 40K promoter. rV-B7-1/ICAM-1 is a recombinant vaccinia virus that contains the murine B7-1 gene under control of the synthetic early/late (sE/L) promoter (ref. 8) and the murine ICAM-1 gene under control of the 40K promoter. rV-B7-1/ICAM-1/LFA-3 is a recombinant vaccinia virus that contains the murine LFA-3 gene under control of the vaccinia 30K (M2L) promoter) (ref. 9), the murine ICAM-1 gene under control of the vaccinia 13 promoter (ref. 10), and the murine B7-1 gene under control of the synthetic early/late (sE/L) promoter. rF-CEA/B7-1/ICAM-1/LFA-3 is a recombinant fowlpox virus that contains the human carcinoembryonic antigen (CEA) gene under control of the 40K promoter, the murine B7-1 gene under control of the sE/L promoter, the murine LFA-3 gene under control of the I3 promoter, and the murine ICAM-1 gene under control of the vaccinia 7.5K promoter (ref. 11). Non-recombinant vaccinia virus was designated V-Wyeth, while non-recombinant fowlpox virus was designated WT-FP.

### INTERNALLY CITED REFERENCES

(1) Wingren, A. G., Parra, E., Varga, M., Kalland, T., Sjogren, H. O., Hedlund, G., & Dohlsten, M. (1995) Crit Rev Immunol 15, 235-53.

(2) Hodge, J. W., Abrams, S., Schlom, J. & Kantor, J. A. (1994) Cancer Res 54, 5552-5.

(3) Uzendoski, K., Kantor, J. A., Abrams, S. I., Schlom, J. & Hodge, J. W. (1997) Hum Gene Ther 8, 851-60.

(4) Lorenz, M. G. O., Kantor, J. A., Schlom, J. & Hodge, J. W. (1998) Human Gene Therapy In Press.

(5) Gritz, L., Destree, A., Cormier, N., Day, E., Stallard, V., Caiazzo, T., Mazzara, G. & Panicali, D. (1990) J Virol 64, 5948-57.

(6) Mazzara, G. P., Destree, A. & Mahr, A. (1993) Methods Enzymol 217, 557-81.

(7) Jenkins, S., Gritz, L., Fedor, C. H., O'Neill, E. M., Cohen, L. K. & Panicali, D. L. (1991) AIDS Res Hum Retroviruses 7, 991-8.

(8) Chakrabarti, S., Sisler, J. R. & Moss, B. (1997) BioTechniques 23, 1094-7.

(9) Perkus, M. E., Piccini, A., Lipinskas, B. R. & Paoletti, E. (1985) Science 229, 981-4.

(10) Schmitt, J. F. & Stunnenberg, H. G. (1988) J Virol 62, 1889-97.

(11) Venkatesan, S., Baroudy, B. M. & Moss, B. (1981) Cell 25, 805-13.

(12) Chakrabarti, S., Brechling, K. and Moss, B. (1985) Mol. Cell. Biol. 5:3403-3409.

(13) Chakrabarti, S., Sisler, J. R., and Moss, B. (1997) BioTechniques 23:1094-1097.

(14) Gillard, S., Spehner, D., Drillien, R., and Kirn, A. (1986) Proc. Natl. Acad. Sci. USA 83:5573-5577.

(15) Morgan, J. R., and Roberts, B. E. (1994) J. Virol. 51:283-297.

(16) Smith, K., Stallard, V., Ross, J., Hart, C., Cormier, N., Cohen, L., Roberts, B., and Payne, L. (1993) Vaccine 11:43-53.

(17) Venkatesan, S., Baroudy, B. M., and Moss, B. (1981) Cell 125:805-813.

### IV. The Different Aspects of the Present Invention

The present invention employs the prepared TRICOM viral construct described herein as the means for generating a genetically altered neoplastic cell; and then utilizing the genetically altered tumor cell in a variety of different conditions and settings as an immunostimulatory agent against a tumor of interest in living mammalian subject.

Note that the prepared TRICOM viral construct may be used to transfect and transduce a tumor cell of interest under in-vivo, and/or ex-vivo and/or in-vitro conditions. Also, it will be recognized and appreciated that the TRICOM vector transfection and transduction of tumor cells can be performed either prophylactically as an in-vivo method of vaccination and prophylactic immunization to prevent the generation in the future of a lymphoma or leukemia in a living mammalian subject; or therapeutically as an in-vivo method of therapeutic treatment effective against a medically diagnosed, pre-existing lymphoma or leukemia then located within in a living mammalian subject. Each of these aspects comprising the invention as a whole will be individually described and delineated.

The Composition of Matter Aspects

The intended consequence and desired result of infecting a lymphoma cell or leukemia cell with the prepared TRICOM viral construct is to generate a genetically altered tumor cell, which subsequently may be employed as an immunostimulatory agent against a tumor of interest, either prophylactically or therapeutically. Thus, the genetically modified neoplastic cell is a unique composition of matter as such; is definable also as a prophylactic vaccine via the method of its manufacture; and is also a genetically altered cell preparation useful as a therapeutic anti-tumor agent to treat a pre-existing tumor clinically in-vivo.

The composition of matter aspects for the purposefully generated and genetically modified tumor cell is definable in a first instance as follows:

A genetically altered neoplastic cell useful as an immunostimulatory agent against a lymphoma or leukemia of interest, said genetically altered neoplastic cell comprising: a cell of mammalian origin which is representative of the tumor cells constituting a lymphoma or a leukemia of interest; a genetically altered genome including at least one extra nucleotide segment comprising a viral vector and not less than one DNA sequence encoding molecule B7.1, intracellular adhesion molecule-1 (ICAM-1) and leukocyte function-associated antigen-3 (LFA-3) as specific products; the capacity to express molecule B7.1, intracellular adhesion molecule-1 (ICAM-1) and leukocyte function-associated antigen 3 (LFA-3) as discrete products and functional costimulatory molecules; and the capability to interact with and to activate CD4+ and CD8+ T-cell lymphocytes in-situ.

Note the essential requirements and characteristic features of the genetically modified cellular composition: first, the tumor cell is of mammalian origin and must exemplify and represent a lymphoma or leukemia of choice. However, the actual source of origin for the mammalian tumor cell may be ex-vivo, in-vivo, or in-vitro. Second, the genetically altered genome of the mammalian tumor cell must be the consequence of an infection and transduction of the native genome by a TRICOM viral vector. Third, the genetically modified genome of the tumor cell has the capacity to express not less than three different exogenous genes or DNA sequences as discrete products and functional costimulatory molecules. These are the B7.1, ICAM-1, and LFA-3 molecules. Fourth, the genetic modifications to the native genome have created a new capacity for the tumor cell—the ability to interact with and to activate CD4+ and CD8+ lymphocytes in-situ.

These genetically modified characteristics and features for the tumor cell constitute not only the essential requirements of the present invention, but also identify the novel and unforeseen attributes and capacities which are shared in common by all embodiments and formats constituting the subject matter as a whole which is the present invention. These requisite structural features and recited attributes are reflected and restated by the other aspects and alternative recitations for the genetically modified tumor cell.

Thus, a second aspect of the essential cellular material is recognized as being a product-by-process result which provides a plurality of such modified tumor cells as a fabricated preparation. This manufactured product is a prophylactic vaccine; and this prepared vaccine is used in-vivo to prevent the generation of a particular lymphoma or leukemia within the body of the living mammalian subject. The product-by-process vaccine is therefore delineated as follows:

A genetically altered neoplastic cell preparation useful as a prophylactic vaccine in-vivo to prevent the generation of a lymphoma or leukemia within the body of a living mammalian subject, said genetically altered neoplastic cell preparation comprising: a plurality of transduced neoplastic cells of mammalian origin which are representative of the tumor cells to be prevented from being generated within the body of the living mammalian subject and have the capability to interact with and to activate CD4+ and CD8+ T-cell lymphocytes in-situ, said transduced neoplastic cells (i) being transduced with a viral vector carrying not less than one DNA sequence encoding molecule B7.1, intracellular adhesion molecule-1 (ICAM-1) and leukocyte function-associated antigen-3 (LFA-3); and (ii) overexpressing molecule B7.1, intracellular adhesion molecule-1 (ICAM-1) and leukocyte function-associated antigen-3 (LFA-3) as discrete peptides and functional costimulatory molecules.

In addition, the same genetically altered neoplastic cell preparation may also be used as an anti-tumor agent therapeutically in-vivo to treat clinically an identified, pre-existing lymphoma or leukemia located at an anatomic position within the body of a living mammalian subject. Under these circumstances, it is intended and expected that the particular lymphoma cells or leukemia cells are removed from the tumor then existing within the body of the subject; that these ex-vivo obtained tumor cells are then infected and transduced by the prepared TRICOM vector construct intentionally and purposefully under aseptic medical conditions; and that the genetically modified tumor cells are then returned in a clinically acceptable manner via an appropriate mode of administration to the appropriate location and anatomic site in the living subject as an autologous cell preparation.

Accordingly, this alternative aspect and delineation is as follows:

A genetically altered neoplastic cell preparation useful as a therapeutic anti-tumor agent in-vivo to treat clinically a pre-existing lymphoma or leukemia within the body of a living mammalian subject, said genetically altered neoplastic cell preparation comprising: a plurality of transduced neoplastic cells of mammalian origin which are representative of the tumor cells in the pre-existing tumor within the body of the living mammalian subject and have the capability to interact with and to activate CD4+ and CD8+ T-cell lymphocytes in-situ, said transduced neoplastic cells (i) being transduced with a viral vector carrying not less than one DNA sequence encoding molecule B7.1, intracellular adhesion molecule-1 (ICAM-1) and leukocyte function-associated antigen-3 (LFA-3); and (ii) overexpressing molecule B7.1, intracellular adhesion molecule-1 (ICAM-1) and leukocyte function-associated antigen-3 (LFA-3) as discrete peptides and functional costimulatory molecules.

The Method of Manufacture Aspects

The present invention intends and relies upon conventionally known techniques, media, reagents, and apparatus to achieve an infection and transduction of neoplastic (or tumor) cells via a prepared TRICOM vector construct as described. The reader is directed to the empirical experiments provided hereinafter as specific instances and examples of the transfection and transduction techniques and protocols. In addition, a variety of viral vector transfections and transduction methods have been previously developed in this field; and constitute a well documented and publicly reported scheme and series of processes well established in the scientific literature. Merely illustrative of such transfection and transduction techniques are the methods described in: Genetic Engineering, Principles And Methods (J. K. Setlow & A. Hollaender, editors), Plenum Press, Volumes 1-3, 1979; Genetic Analysis: Principles, Scope and Objectives (John R. S. Fincham), Blackwell Science Ltd., 1994; Molecular Cloning, A Laboratory Manual, (T. Maniatis, E. F. Fritsch & J. Sambrook), Cold Spring Harbor Laboratory, 1982; and Recombinant DNA Methodology II (Ray Wu, editor), Academic Press, 1995. The texts of each publication is expressly incorporated by reference herein.

The method of manufacture aspects concerning the present invention are thus two:

The first is a method for making a genetically altered neoplastic cell useful as an agent against a tumor of interest, said method comprising the steps of: obtaining a neoplastic cell of mammalian origin which is representative of the tumor cells constituting a tumor of interest; altering the genome of said neoplastic cell by introduction of at least one extra nucleotide segment comprising a viral vector and not less than one DNA sequence encoding molecule B7.1, intracellular adhesion molecule-1 (ICAM-1) and leukocyte function-associated antigen-3 (LFA-3) as specific products; and (ii) allowing said altered genome of said B lymphoma cell to express molecule B7.1, intracellular adhesion molecule-1 (ICAM-1) and leukocyte function-associated antigen-3 (LFA-3) as discrete products and functional costimulatory molecules.

The second mode of manufacture is a method for making transduced tumor cell preparation useful as an immunostimulatory agent in-vivo effective against a tumor of interest, said transduced tumor cell preparation method comprising the steps of: obtaining a plurality of neoplastic cells from the body of the living mammalian subject which are representative of the tumor of interest; transducing said neoplastic cells with a viral vector carrying not less than one DNA sequence encoding molecule B7.1, intracellular adhesion molecule-1 (ICAM-1) and leukocyte function-associated antigen-3 (LFA-3); and (ii) allowing said transduced B lymphoma cells to overexpress molecule B7.1, intracellular adhesion molecule-1 (ICAM-1) and leukocyte function-associated antigen-3 (LFA-3) as discrete peptides and functional costimulatory molecules.

The Method of Use Aspects

The subject matter as a whole which comprises the present invention envisions and intends that two separate use applications and two markedly different beneficial outcomes be achieved. The first is a prophylactic application and result; the second is an autologous therapeutic course of treatment. Each will be described individually.

Prophylactic Immunization:

A first use and application is in-vivo prophylaxis to prevent the future generation of a neoplasm in the body of a living subject. In this instance and application, the neoplastic cells are exogenous and foreign to the intended living recipient; and these foreign tumor cells are purposely infected and transduced in-vitro to yield genetically modified tumor cells now able to overexpress not less than three requisite antigens on their cell surfaces—the B7.1, ICAM-1 and LFA-3 molecules.

These genetically modified neoplastic cells thus constitute and provide a vaccine comprised of genetically altered exogenous tumor cells, which is then to be administered to a living recipient as an immunostimulatory preparation. The in-vivo administered vaccine will then interact with and activate CD4+ and CD8+ T-cell lymphocytes in-vivo within the body of the living subject; and thus create an in-vivo prophylaxis and cytoimmune prevention of tumor generation in the future for that living subject.

Accordingly, this prophylactic immunization aspect is delineated as:

A method of in-vivo prophylaxis to prevent the generation of a tumor in a living mammalian subject, said in-vivo prophylaxis method comprising the steps of: obtaining a vaccine comprising a plurality of transduced neoplastic cells of mammalian origin which are representative of the tumor to be prevented from generating within the body of the living mammalian subject, wherein said transduced neoplastic cells (i) have been transduced with a viral vector carrying not less than one DNA sequence encoding molecule B7.1, intracellular adhesion molecule-1 (ICAM-1) and leukocyte function-associated antigen-3 (LFA-3); and (ii) overexpressing B7.1, intracellular adhesion molecule-1 (ICAM-1) and leukocyte function-associated antigen-3 (LFA-3) as discrete peptides and functional costimulatory molecules; administering said vaccine to the body of the living mammalian subject; and allowing said transduced neoplastic cells of said administered vaccine to interact with and to activate CD4+ and CD8+ T-cell lymphocytes in-vivo within the living mammalian subject.

Therapeutic Treatments.

The second and markedly different aspect is as an autologous anti-tumor agent and effective therapeutic treatment against a medically recognized and clinically diagnosed pre-existing tumor in the body of a living mammalian subject. In this instance, the living subject has been identified previously as being afflicted with a tumor of recognized type which presently lies at one or more anatomic sites within the body of the patient. The neoplastic cells to be infected and transduced via a TRICOM vector construct are thus taken directly from the patient in question—as an ex-vivo biopsy specimen or by other medically acceptable ex-vivo means; and these ex-vivo neoplastic cells, after being intentionally transduced and genetically altered, are then administered via an appropriate route and means back to the original donor and tumor-carrying patient as an autologous cellular preparation now able to serve and function as an anti-tumor agent.

This therapeutic treatment method offers several highly desirable benefits and advantages. First, the transduced neoplastic cells serving as the anti-tumor agent are specific for the tumor then existing in-situ within the patient. Thus, when administered as an immunostimulatory preparation in-vivo, the particular tumor cell surface antigens will become specifically recognized by the activated T-lymphocytes; and the activated T-cells will function against the tumor mass in-vivo with a greater degree of cytolytic specificity. Second, the transduced neoplastic cells are not foreign or exogenous to the patient in question; and thus constitute an autologous agent which is more biocompatible to the recipient. Third, the overexpressed surface antigens—the B7.1, ICAM-1 and LFA-3 molecules—are functional costimulatory molecules acting at the cell surface. These co-stimulatory molecules act efficaciously in-vivo to activate CD4+ and CD8+ lymphocytes; and via such in-vivo interaction and activation provoke a cytological immune response which acts specifically against the preexisting tumor in-situ.

Accordingly, the therapeutic treatment is recited as follows:

A method of in-vivo therapeutic treatment effective against a pre-existing tumor in a living mammalian subject, said in-vivo therapeutic treatment method comprising the steps of: obtaining a cell preparation comprising a plurality of transduced neoplastic cells of mammalian origin which are representative of the pre-existing tumor within the body of the living mammalian subject, wherein said transduced neoplastic cells (i) have been transduced with a viral vector carrying not less than one DNA sequence encoding molecule B7.1, intracellular adhesion molecule-1 (ICAM-1) and leukocyte function-associated antigen-3 (LFA-3); and (ii) overexpress B7.1, intracellular adhesion molecule-1 (ICAM-1) and leukocyte function-associated antigen-3 (LFA-3) as discrete peptides and functional costimulatory molecules; administering said cell preparation to the body of the living mammalian subject as an anti-tumor agent; and allowing said irradiated, transduced B lymphoma cells of said administered cell preparation to interact with and to activate CD4+ and CD8+ T-cell lymphocytes in-vivo within the living mammalian subject.

### V. Experiments and Empirical Data

To demonstrate the merits and value of the present invention, a series of planned experiments and empirical data are presented below. It will be expressly understood, however, that the experiments described and the results provided are merely the best evidence of the subject matter as a whole which is the invention; and that the empirical data, while limited in content, is only illustrative and representative of the scope of the invention envisioned and claimed.

Materials And Methods:

Animals

Six- to 8-week-old female BALB/c mice were purchased from Harlan-Sprague-Dawley (San Diego, Calif.), and were housed at the Laboratory Animal Facility at Stanford University Medical Center (Stanford, Calif.). All experiments were conducted in accordance with the Stanford University Animal Facility Care guidelines.

Cell Lines

A20 is a BALB/c B cell lymphoma line expressing major histocompatibility complex class I and II H-2d molecules and was obtained from the American Type Culture Collection (ATCC, Rockville, Md.). Tumor cells were cultured in RPMI 1640 medium (Gibco BRL, Rockville, Md.) supplemented with 10% heat-inactivated fetal calf serum (“FCS”) (Hyclone, Logan, Utah), 100 U/mL penicillin and 100 μg/mL streptomycin (both from Gibco BRL), and 50 μM 2-mercaptoethanol (Sigma, St. Louis, Mo.); and is further referred to as “complete media”. Cells were grown in suspension culture at 37° C., in 5% CO₂. Hybridomas, GK1.5 [anti-CD4], 53-6.72 [anti-CD8], and H22-15-5 [rat IgG2], as well as the P815 mastocytoma cell line were obtained from ATCC, and were cultured in complete media.

In Vitro Transduction of Tumor Cells with Recombinant Fowlpox Viruses

The recombinant fowlpox virus encoding the murine costimulatory molecules B7-1, ICAM-1, and LFA-3 (designated TRICOMO was provided by Therion Biologics Corporation (Cambridge, Mass.). A non-recombinant wild-type fowlpox virus (WT-FP) was used as a negative control. A20 cells cultured in complete media, but containing only 2% FCS, were infected with the TRICOM virus at a multiplicity of infection (MOI) of 100 for 18 hours.

After viral infection, the cells were harvested and the expression of costimulatory molecules was assessed by flow cytometry. For this purpose, an aliquot of cells was washed twice in PBS-1% bovine serum albumin plus 0.05% sodium azide, and stained for 30 minutes on ice with a panel of phycoerythrin-conjugated monoclonal antibodies (MAbs) specific for murine B7.1 (CD80), B7.2 (CD86), ICAM-1 (CD54), and CD48 (the mouse homologue of LFA-3) [all from PharMingen, San Diego, Calif.]. 4-1BB Ligand (4-1BBL) expression was detected using a MAb specific for murine 4-1BBL (rat IgG2a) and a fluorescein isothiocyanate-conjugated mouse-anti-rat IgG1 MAb (both from PharMingen). Appropriate isotype controls were used in all experiments. After incubation, the cells were washed and then fixed with 2% paraformaldehyde, and the cells were analyzed using a Becton Dickinson FACScan (Mountain View, Calif.) with the CellQuest software.

Vaccination and Tumor Challenge Experiments

The A20 tumor cells were thawed from a common frozen stock; and passaged in vitro in complete media for 4 days before use. On the day of tumor challenge, the cells were washed 3 times in RPMI-1640 (no supplements); and them diluted to the appropriate concentration in RPMI. Groups of mice (10 per group) received 2×10⁵ cells in a volume of 0.2 mL, subcutaneously (s.c.) in one flank.

For prophylactic experiments, mice were vaccinated twice s.c. at 2-week intervals with 1×10⁶ A20 cells previously infected in vitro with TRICOM or with control WT-FP virus. After the infection, the transfected cells were washed extensively with RPMI-1640, and irradiated (5,000 cGy) immediately before injection.

One week after the last vaccine, animals were challenged with live tumor cells as described above. Animals were followed daily for survival. Survival analysis was performed using Prism software (GrapPad, San Diego, Calif.), and statistical differences in survival were calculated using the long-rank test.

Treatment of Pre-Existing Tumors

For therapeutic experiments, mice were first injected s.c. with 2×105 A20 cells in one flank. On days 5 and 11 after tumor challenge, the mice received two s.c. injections (on the opposite flank). of 1×10⁶ irradiated tumor cells previously transduced with TRICOM, or WT-FP virus.

Tumors were measured three times weekly in two dimensions (length and width) with a caliper; and tumor volumes were calculated according to the formula: (width)²×length×0.52. Tumor volumes are reported as mean mm³±standard error. P values were determined by using a two-tailed t test; and a P value of less than 0.05 was considered to be statistically significant. Animals were followed for survival as described above.

In some experiments, mice with established tumors were treated by intratumoral injection of fowlpox viruses. For this purpose, tumor cells (10⁵) were injected s.c. When tumors could be palpated (5-6 mm in maximal diameter; day 22), they were injected with 10⁸ pfu (in 100 μl in PBS) of either TRICOM; or a fowlpox recombinant vector producing murine granulocyte/macrophage-colony stimulating factor (GM-FP, provided by Therion Biologics Corporation); or a combination of both of these. WT-FP was used as a control virus. In each instance, a second dose of virus was given 5 days apart from the first. Tumor volumes were measured and reported as described above.

T-cell Depletion Experiments

Mice (10 per group) were vaccinated twice (2 separate occasions) with irradiated, TRICOM-transduced tumor cells; and then challenged one week after the second vaccination with live tumor cells. Animals were depleted of CD4+ and CD8+ cells by intraperitoneal injection of anti-CD4 (GK1.5 hybridoma) or anti-CD8 (53-6.72 hybridoma) ascitic fluid.

Antibodies (200 μl of diluted ascitic fluid/dose) were injected on days −3, −2, −1, and 0 before the tumor challenge; and then every other day (post-tumor challenge) for a week; and then was followed by four weekly injections. A group of mice received the irrelevant rat IgG2 antibody (H22-15-5 hybridoma) as a control. These depletions conditions were validated by flow cytometry analysis of splencytes using PE-conjugated MAbs anti-CD4 (Caltag, Burlingame, Calif.), anti-CD8β.2 (clone 53-5.8; PharMingen); 99% of the relevant cell subset was depleted, whereas all other subsets remained within normal levels.

Tumor-Specific Cytotoxicity Assays

Ten days after the second and last vaccination, splenocytes and lymph nodes were removed and isolated from 2 representative mice of each group; “pooled”; and then restimulated at 5×10⁶ cells/mL with irradiated (5,000 cGy) A20 cells (1×10⁶/mL) for 6 days. IL-2 (10 U/mL; Chiron, Emeryville, Calif.) was added to the cell cultures on day 3. Viable cells were harvested and then tested in a 4-hour standard ⁵¹Cr release assay for the ability to lyse A20 cells.

Briefly, in this analytical test, ⁵¹Cr labeled A20 or P815 cells were incubated with effector cells at different effector:target ratios in triplicate wells; and ⁵¹Cr release was determined by analyzing the supernatants in a gamma counter (Wallac, Turku, Finland). The percentage of specific release was calculated according to the formula: 100×[(experimental release-spontaneous release)/(maximal release-spontaneous release)]. Spontaneous release and maximum release were obtained from wells containing target cells incubated in medium alone or in 1% Triton-X, respectively.

ELISA for Detection of Anti-Idiotype Antibodies

Serum from vaccinated mice was added to 96-well MaxiSorp plates (Nunc, Naperville, Ill.) coated with purified recombinant single chain Fv A20 protein (100 μg/mL) (kindly provided by H. Veelken, Freiburg University, Germany). Anti-Idiotype antibody was detected by using HRP-conjugated goat anti-mouse IgG (Caltag). Serum known to contain anti-A20 idiotype antibodies served as positive control. Absorbance was determined at 405 nm using a Vmax microplate reader (Molecular Devices, Menlo Park, Calif.).

Empirical Results:

### Experimental Series 1

In vitro expression of costimulatory molecules on TRICOM-transduced A20 cells To demonstrate that upregulation of costimulatory molecules could be induced in the A20 system, the A20 cells were infected either with TRICOM or the WT-FP virus; and the cell surface expression of B7-1, ICAM-1, and LFA-3 was assessed by flow cytometry. The results are graphically illustrated by FIGS. 1A-1D respectively.

FIG. 1 as a whole shows the upregulation of costimulatory molecules on A20 cells after transduction with TRICOM. A20 lymphoma B cells were infected either with TRICOM (black histogram) or wild-type fowlpox vectors (white histogram) at a multiplicity of infection of 100 for 18 hrs. Cells were analyzed by flow cytometry. Broken line histogram represents cells stained with an isotype control mAb. In addition to upregulation of B7-1 (FIG. 1A), ICAM-1 (FIG. 1B) and LFA-3 (FIG. 1C) encoded by the TRICOM vector, increased expression of the molecule 4-1BBL (FIG. 1D) is also noted. Other abbreviations used: TRICOM=triad of costimulatory molecules; ICAM-1=intercellular adhesion molecule-1; LFA-3=leukocyte function-associated antigen-3; 4-1BBL=4-1BB ligand.

Thus, as shown by FIG. 1 as a whole, WT-FP virus infected cells lack expression of B7-1, and expressed moderate levels of ICAM-1, and LFA-3. However, after transduction with TRICOM, 100% of the cells become positive for B7-1 and expressed higher levels of both ICAM-1 (mean fluorescence intensity [MFI]; WT-FP vs TRICOM, 56.5% vs 140.5, respectively) and LFA-3 (166 vs 372). Interestingly, the T-cell costimulatory molecule 41BBL, which is expressed at very low levels by WT-FP-infected A20 cells, was highly upregulated after TRICOM infection (7% vs 75% positive cells). However, the expression of other surface molecules such as B7-2 and Fas was not affected (data not shown).

### Experimental Series 2: In Vivo Antitumor Effect of Irradiated TRICOM-Transduced Tumor Cells

As a way to stimulate systemic immunity against a lymphoma, irradiated tumor cells were transduced to express high levels of B7-1, ICAM-1, and LFA-3. Initially, an analysis was made to determine if irradiated TRICOM-transduced tumor cells could confer systemic immunity against lymphoma. For this purpose, mice were twice vaccinated with irradiated A20 tumor cells that were previously transduced in vitro with TRICOM; and then the vaccinated mice were challenged with a lethal dose of parental A20 tumor cells. The results are graphically illustrated by FIGS. 2A and 2B respectively.

FIG. 2 as a whole demonstrates the survival of mice vaccinated with irradiated TRICOM-transduced tumor cells. FIG. 2A illustrates those mice (n=10 per group) vaccinated twice (two weeks apart) s.c. with 10⁶ irradiated (5,000 cGy) A20 cells, which were previously transduced in vitro with TRICOM (□) or wild-type fowlpox (WT-FP) (Φ) viruses. One week after the second vaccination the mice were challenged with 2×10⁵ parental A20 tumor cells s.c. and followed for survival. A group of naive mice (♦) received tumor challenge only.

In comparison, FIG. 2B illustrates those mice vaccinated with TRICOM-transduced tumor cells (□) that rejected the tumor and were then re-challenged with another equal dose of parental A20 tumor cells. As a control, a group of naive mice (♦) received the tumor challenge only. Abbreviations: TRICOM=triad of costimulatory molecules.

Thus, as shown by the data illustrated by FIG. 2A, 80% of the animals survived and remained tumor-free at the end of the experiment (P<0.001 vs WT-FP). In contrast, mice vaccinated with the tumor cells transduced with WT-FP virus were not protected (P=0.6 vs no vaccine).

As a followup to test whether vaccination with TRICOM-transduced tumor cells could induce long-term immunity against the parental tumor, animals that rejected the tumor (n=8) were re-challenged at 110 days after the initial challenge. Seven out of 8 mice were tumor-free, while all control animals (n=10) developed disease (FIG. 2B).

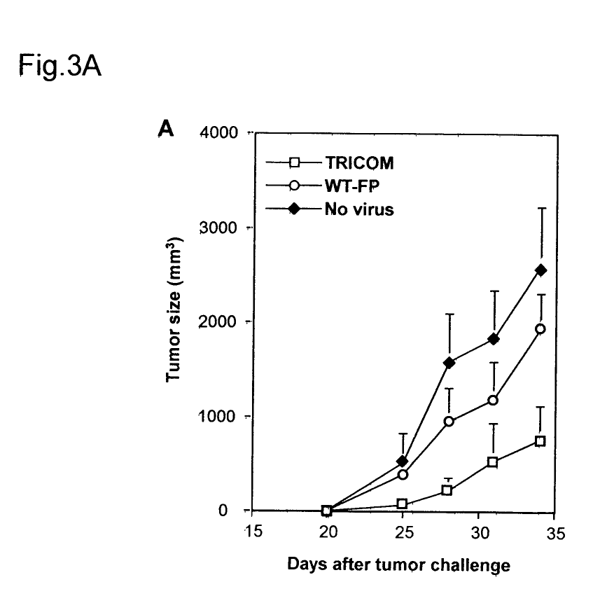

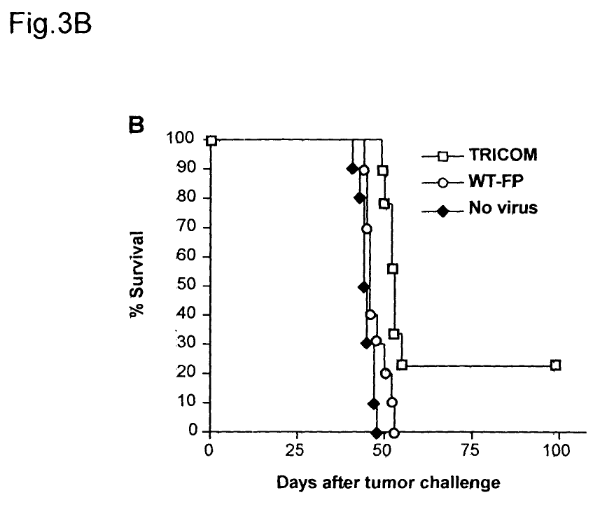

Subsequently, the capacity of the vaccine to stimulate immunity in animals after they had tumor cells growing was evaluated. Mice were first challenged with 2×10⁵ parental A20 cells; and then treated on days 5 and 11 after tumor challenge with 10⁶ irradiated TRICOM-transduced A20 tumor cells. The results are graphically shown by FIGS. 3A and 3B respectively.

FIG. 3 as a whole graphically shows the survival of mice with pre-existing tumors treated with irradiated TRICOM-transduced tumor cells. Groups of 10 mice were injected with 2×10⁵ parental A20 tumor cells s.c. On days 5 and 11, mice were treated with 10⁶ irradiated A20 cells transduced in vitro with TRICOM (□), wild-type fowlpox (WT-FP) (Φ) viruses or uninfected A20 cells (♦); and the size of the tumor shown by FIG. 3A and survival rate shown by FIG. 3B was monitored. Abbreviations: TRICOM=triad of costimulatory molecules.

Clearly, tumor growth was slower in those animals treated with TRICOM-transduced tumor cells, as compared to animals treated with WT-FP-transduced cells (758±218 mm³ vs 1945±152 mm³, respectively; day 34; P=0.0017) [FIG. 3A]. More importantly, animals receiving TRICOM-transduced tumor cells had a survival advantage as compared to control animals (20% vs 0%, P<0.0001) [FIG. 3B].

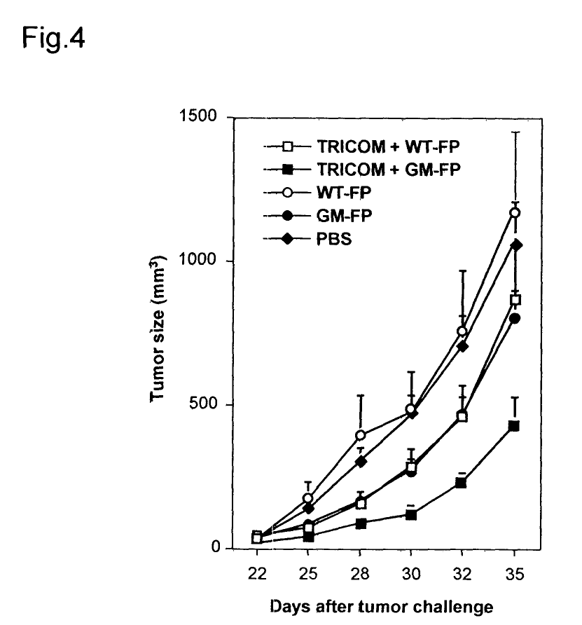

In another test series, mice with pre-established tumors were treated by two intratumoral injections (5 days apart) of recombinant fowlpox viruses (TRICOM, GM-FP, a combination of both, or WT-FP) or PBS alone. The results are graphically shown by FIG. 4.

FIG. 4 illustrates the consequence of treatment of mice with established tumors with intratumoral injection of recombinant fowlpox viruses. Mice (10 per group) were administered 10⁵ of parental A20 tumor cells s.c. When the tumors were palpable, they were injected twice, but 5 days apart, with 10⁸ pfu of TRICOM virus (Γ); or GM-FP virus (M), or a combination of both of these; or wild-type fowlpox virus (WT-FP) (Φ). A control group of mice received treatment with PBS ( ) only. The size of the tumor in each instance was assessed using a caliper and reported as the mean mm3±standard error. Other abbreviations used: TRICOM=triad of costimulatory molecules; GM-FP=fowlpox recombinant vector producing granulcyte/macrophage-colony stimulating factor.

As evidenced by the data of FIG. 4, although the TRICOM virus alone did not significantly inhibit the growth of the tumors (875±211 vs 1192±262; day 35; P=0.36), mice treated with the combination of TRICOM virus plus GM-FP virus had smaller tumors compared to those mice treated with a similar dose of WT-FP virus (436±96 vs 1192±262; day 35; P=0.018). However, there was no survival advantage for those mice treated with the combination of recombinant viruses (data not shown).

### Experimental Series 3: Cellular and Humoral Immune Responses in Vaccinated Mice

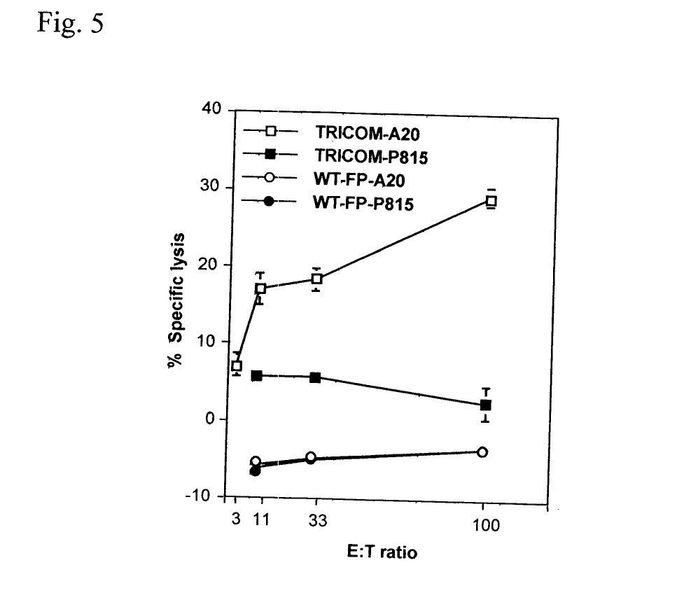

To examine the generation of cytotoxic T-lymphocytes (CTL) responses against parental tumor cells, lymph node and splenocytes from mice vaccinated with irradiated TRICOM-transduced tumor cells were cultured with irradiated parental A20 tumor cells for 6 days. The results are graphically illustrated by FIG. 5.

FIG. 5 demonstrates and evidences the cytotoxic T lymphocyte activity induced by irradiated tumor cells transduced with TRICOM. Lymph node and spleen cells from mice vaccinated with irradiated tumor cells transduced with TRICOM or wild-type fowlpox (WT-FP) viruses were restimulated in vitro for 6 days with irradiated (5000 cGy) parental A20 tumor cells. Cytotoxic activity against A20 or P815 was then measured using the standard 4-hour ⁵¹Cr release assay. The data of FIG. 5 is presented as mean specific lysis of triplicate values (%)±standard deviation at different E:T ratios. TRICOM-vaccinated vs. A20 (Γ) or P815 ( ) target cells. WTFP-vaccinated vs. A20 (Φ) or P815 (M) target cells. Abbreviations: TRICOM=triad of costimulatory molecules.

Thus, as shown by the data of FIG. 5, effector cells from mice vaccinated with tumor cells previously transduced with TRICOM were able to lyse parental A20 tumor cells. Note that this lysis was tumor specific, since the same effector cells were not able to lyse the syngeneic tumor cell line P815. Also, no significant CTL activity was detected in mice vaccinated with tumor cells previously infected with the WT-FP virus.

As a followup study, evidence was obtained that empirically demonstrated a humoral response against the A20 idiotype protein in mice vaccinated with irradiated TRICOM-transduced A20 cells. In contrast to serum from mice vaccinated with recombinant A20 idiotype protein conjugated to keyhole limpet hemocyanin, no anti-idiotype antibodies were detected in the serum from mice collected 5 days after the second vaccine (data not shown).

### Experimental Series 4: Involvement of CD4+ and CD8+ T Cells in the Antitumor Effect of Irradiated TRICOM-Transduced Tumor Cells

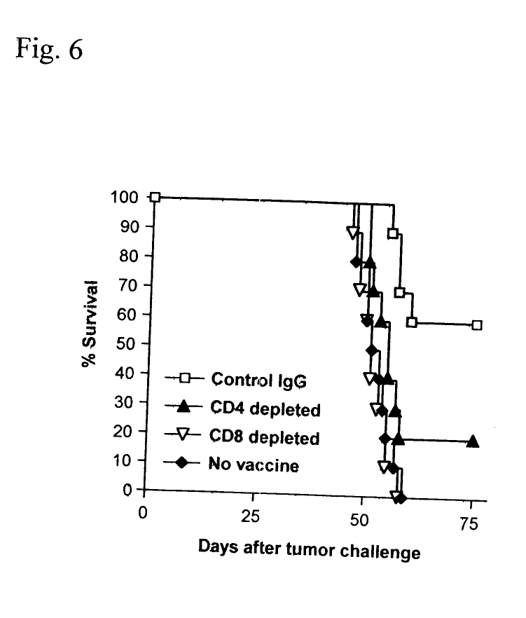

This experimental study characterized the effector cells involved in the antitumor effect that vaccination with tumor cells transduced with TRICOM. After the vaccine was administered, and before the tumor challenge, mice were depleted of T cells by the administration of MAbs. Antibodies were also given after the tumor challenge, to ensure continued depletion of the relevant cell subsets. The results and data are graphically illustrated by FIG. 6.

FIG. 6 demonstrates the effect of in vivo depletion of T cell subsets on survival following vaccination with TRICOM-transduced tumor cells. Groups of 10 mice were vaccinated twice, 2 weeks apart s.c. with 10⁶ irradiated A20 cells transduced in vitro with TRICOM, and challenged with 2×10⁵ parental A20 tumor cells 1 week after the last vaccine. Mice were depleted of T cells by i.p. administration of GK 1.5 (anti-CD4) (π) or 53-6.72 (anti-CD8) (σ) antibodies. A group of mice received rat IgG (H22-15-5) as control (Γ). A group of naive mice received tumor challenge only ( ). Abbreviations: TRICOM=triad of costimulatory molecules.

Clearly, as the data of FIG. 6 reveals, both CD4⁺ and CD8⁺ T cells were found to be important for the antitumor effect induced by the vaccine, since tumor-free survival was reduced in those vaccinated animals that were depleted of CD4⁺ and CD8⁺ T cells (P=0.023, and P=0.0001 vs TRICOM, respectively).

Conclusions Supported By The Empirical Results And Data

1. In this study, it has been unequivocally shown that A20 lymphoma cells transduced with TRICOM virus express high levels of the costimulatory molecules B7-1, ICAM-1, and LFA-3; and can be used as a vaccine to promote immunity against wild-type tumor cells. Thus, while parental A20 cells express no B7-1 and express only moderate levels of ICAM-1 and LFA-3 after infection with the TRICOM vector, 100% of the lymphoma cells expressed B7-1, and also expressed increased levels of both ICAM-1 and LFA-3.

2. Mice immunized (vaccinated) with irradiated TRICOM-transduced tumor cells were able to reject a challenge with parental A20 tumor cells. The majority of the vaccinated animals were also able to reject a second challenge with parental A20 tumor cells long after being initially immunized, a result which is consistent with the development of a memory immune response.

3. The increased immunogenicity of TRICOM-transduced A20 cells is also related to the expression of additional T-cell stimulatory molecules not directly encoded by the transgenes of the vector. Thus, the 4-1BBL molecule expressed by an APC, directly activates both CD4⁺ and CD8⁺ T cells; and the 4-1BBL has a synergistic effect with B7-1 at promoting anti-tumor immunity. More importantly, the combination of expressing both B7-1 and 4-1BBL molecules induces in vivo antitumor immunity in the A20 model system.

4. The reported data evidences and demonstrates that the use of tumor cells expressing high levels of costimulatory molecules can induce significant antitumor effects in living subjects that have a pre-existing lymphoma. The effect of this approach when the TRICOM virus is injected directly into the tumor was also studied. In this more stringent scenario, the tumors injected with TRICOM virus had a trend towards a slower growth, but this resulting effect did not reach a degree of statistical significance. This slower growth result may be explained, in part, by the relatively low dose of TRICOM virus used, since it has been previously shown that the effect of an intratumoral administration of recombinant viruses expressing immune stimulatory proteins is largely dose dependent. It is particularly noted, however, that a significant inhibition of in-vivo tumor growth was found to occur when intratumoral administration of the TRICOM virus was combined with a second fowlpox virus encoding murine GM-CSF.

5. The mechanism of inducing tumor protection after vaccination with irradiated TRICOM-transduced A20 tumor cells was also investigated. It was found that a requirement for T lymphocytes exists in the induction of an antitumoral effect. In particular, it is noted that in vivo depletion of CD8⁺ T cells completely abrogated the development of a protective immune response. CD4⁺ T cells were also found to be involved in the antitumoral effect. These results support the in vitro studies showing that mice vaccinated with tumor cells transduced with TRICOM (but not with a control virus) generate effector T cells able to specifically recognize parental A20 tumor cells. This may be related with the fact that tumor A20 cells express both MHC class I and II molecules, thus making the MHC antigens potential targets for both CD4⁺ and CD8⁺ T cells. Alternatively, CD4⁺ T cells may be involved indirectly by providing help in maintaining CD8⁺ effector T cells.

6. While cellular immune mechanisms appear to be responsible for the antitumoral effect observed with the use of cellular vaccines, the role of a humoral (antibody) response against antigens expressed by the tumor cells is less clear. Although one cannot rule out the existence of circulating antibodies against unknown tumor antigens, the data of the experiments suggest that antibodies directed against one known B-cell lymphoma tumor antigen (the idiotype) does not play a substantive role in the antitumor protection observed.

The present invention is not to be limited in form nor restricted in scope except by the claims appended hereto.

What we claim is:

1. A genetically altered neoplastic cell useful as an immunostimulatory agent against a tumor of interest, said genetically altered neoplastic cell comprising:
a neoplastic cell of mammalian origin which is representative of the cells constituting a tumor of interest;
a genetically altered genome including at least one extra nucleotide segment comprising a viral vector and not less than one DNA sequence encoding molecule B7.1, intracellular adhesion molecule-1 (ICAM-1) and leukocyte function-associated antigen-3 (LFA-3) as specific products;
the capacity to overexpress molecule B7.1, intracellular adhesion molecule-1 (ICAM-1) and leukocyte function-associated antigen-3 (LFA-3) as discrete products; and
the capability to interact with and to activate CD4+ and CD8+ T-cell lymphocytes in-situ.

2. A genetically altered neoplastic cell preparation useful as a prophylactic vaccine in-vivo to prevent the generation of a tumor within the body of a living mammalian subject, said genetically altered neoplastic cell preparation comprising:
a plurality of transduced neoplastic cells of mammalian origin which are representative of the tumor to be prevented within the body of the living mammalian subject and which have the capability to interact with and to activate CD4+ and CD8+ T-cell lymphocytes in-situ, said transduced neoplastic cells
(i) being transduced with a viral vector carrying not less than one DNA sequence encoding molecule B7.1, intracellular adhesion molecule-1 (ICAM-1) and leukocyte function-associated antigen-3 (LFA-3); and
(ii) overexpressing molecule B7.1, intracellular adhesion molecule-1 (ICAM-1) and leukocyte function-associated antigen-3 (LFA-3) as discrete peptides and functional costimulatory molecules.

3. A genetically altered neoplastic cell preparation useful as a therapeutic anti-tumor agent in-vivo to treat clinically a pre-existing tumor within the body of a living mammalian subject, said genetically altered neoplastic cell preparation comprising:
a plurality of transduced neoplastic cells of mammalian origin which are representative of the cells in the pre-existing tumor within the body of the living mammalian subject and which have the capability to interact with and to activate CD4+ and CD8+ T-cell lymphocytes in-situ, said transduced neoplastic cells
(i) being transduced with a viral vector carrying not less than one DNA sequence encoding molecule B7.1, intracellular adhesion molecule-1 (ICAM-1) and leukocyte function-associated antigen-3 (LFA-3); and
(ii) overexpressing molecule B7.1, intracellular adhesion molecule-1 (ICAM-1) and leukocyte function-associated antigen-3 (LFA-3) as discrete peptides and functional costimulatory molecules.

4. A method for making a genetically altered neoplastic cell useful as an agent against a tumor of interest, said method comprising the steps of:
obtaining a neoplastic cell of mammalian origin which is representative of the cells constituting a tumor of interest;
altering the genome of said neoplastic cell by introduction of at least one extra nucleotide segment comprising a viral vector and not less than one DNA sequence encoding molecule B7.1, intracellular adhesion molecule-1 (ICAM-1) and leukocyte function-associated antigen-3 (LFA-3) as specific products;
allowing said altered genome of said neoplastic cell to express molecule B7.1, intracellular adhesion molecule-1 (ICAM-1) and leukocyte function-associated antigen-3 (LFA-3) as discrete products.

5. A method for making a transduced neoplastic cell preparation useful as an immunostimulatory agent in-vivo effective against a tumor of interest, said method comprising the steps of:
obtaining a plurality of neoplastic cells of mammalian origin which is representative of the tumor of interest;
transducing such neoplastic cells with a viral vector carrying not less than one DNA sequence encoding B7.1, intracellular adhesion molecule-1 (ICAM-1) and leukocyte function-associated antigen-3 (LFA-3); and
allowing said transduced neoplastic cells to overexpress molecule B7.1, intracellular adhesion molecule-1 (ICAM-1) and leukocyte function-associated antigen-3 (LFA-3) as discrete peptides and functional costimulatory molecules.

6. A method of in-vivo prophylaxis to prevent the generation of a tumor in a living mammalian subject, said in-vivo prophylaxis method comprising the steps of:
obtaining a vaccine comprising a plurality of transduced neoplastic cells of mammalian origin which are representative of the tumor to be prevented within the body of the living mammalian subject, wherein said transduced neoplastic cells
(i) have been transduced with a viral vector carrying not less than one DNA sequence encoding molecule B7.1, intracellular adhesion molecule-1 (ICAM-1) and leukocyte function-associated antigen-3 (LFA-3), and
(ii) overexpressing molecule B7.1, intracellular adhesion molecule-1 (ICAM-1) and leukocyte function-associated antigen-3 (LFA-3) as discrete peptides and functional costimulatory molecules;
administering said vaccine to the body of the living mammalian subject; and
allowing said transduced neoplastic cells of said administered vaccine to interact with and to activate CD4+ and CD8+ T-cell lymphocytes in-vivo within the living mammalian subject.

7. A method of in-vivo therapeutic treatment effective against a pre-existing tumor in a living mammalian subject, said in-vivo therapeutic treatment method comprising the steps of:
obtaining a cell preparation comprising a plurality of transduced neoplastic cells of mammalian origin which are representative of the cells in the pre-existing tumor within the body of the living mammalian subject, wherein said transduced neoplastic cells
(i) have been transduced with a viral vector carrying not less than one DNA sequence encoding molecule B7.1, intracellular adhesion molecule-1 (ICAM-1) and leukocyte function-associated antigen-3 (LFA-3), and
(ii) overexpressing molecule B7.1, intracellular adhesion molecule-1 (ICAM-1) and leukocyte function-associated antigen-3 (LFA-3) as discrete peptides and functional costimulatory molecules;
administering said cell preparation to the body of the living mammalian subject as an anti-tumor agent; and
allowing said transduced neoplastic cells of said administered cell preparation to interact with and to activate CD4+ and CD8+ T-cell lymphocytes in-vivo within the living mammalian subject.
